(19) United States
(12) Patent Application Publication
(10) Pub. No.: US 2010/0323957 A1
(43) Pub. Date: Dec. 23, 2010

(54) NOVEL ASSAY FOR INHIBITORS OF EGFR

(75) Inventors: John Kuriyan, Berkeley, CA (US); Xuewu Zhang, Berkeley, CA (US); Natalia Jura, Oakland, CA (US)

Correspondence Address: MORGAN, LEWIS & BOCKIUS LLP (SF), One Market, Spear Street Tower, Suite 2800, San Francisco, CA, 94105, US

(73) Assignee: THE REGENTS OF THE UNIVERSITY OF CALIFORNIA THE OFFICE OF THE PRESIDENT, OAKLAND, CA (US)

(21) Appl. No.: 12/743,218

(22) PCT Filed: Nov. 19, 2008
(86) PCT No.: PCT/US08/84080

§ 371 Date: Aug. 23, 2010

Related U.S. Application Data
(60) Provisional application No. 60/988,963, filed on Nov. 19, 2007.

(51) Int. Cl. A61K 38/16; G01N 33/566; C12N 9/99; A61P 35/00

(52) U.S. Cl. 514/7.5; 435/7.8; 435/184

(57) ABSTRACT

The invention provides methods and compositions for screening for modulators of EGFR activity. In particular, an assay for such modulators is provided, which includes methods of screening for modulators using models of the three dimensional structure of EGFR kinase domains.

### CROSS REFERENCE TO RELATED APPLICATIONS

This application claims the benefit of priority to U.S. Provisional Application No. 60/98,963, filed Nov. 19, 2007, which is hereby incorporated by reference in its entirety.

### FIELD OF THE INVENTION

The invention relates generally to the field of molecular biology, biochemistry, and cell biology of the Epidermal Growth Factor Receptor (EGFR). In particular, the instant invention provides methods and compositions for screening for agents that are able to modulate EGFR. EGFR receptors play critical roles in regulating cell proliferation, differentiation, and migration, and their abnormal activation is associated with a variety of human cancers, including lung, breast, pancreatic, ovarian and prostate cancer. Compositions and methods of the invention can be used to prevent, cure, treat, or ameliorate these cancers as well as other diseases associated with EGFR.

### BACKGROUND INFORMATION

The following is provided as background information only and should not be taken as an admission that any subject matter discussed or that any reference mentioned is prior art to the instant invention. All publications and patent applications herein are incorporated by reference to the same extent as if each individual publication or patent application was specifically and individually indicated to be incorporated by reference.

In multi-cellular organisms, communication between individual cells is essential for the regulation of complex biological processes such as growth, differentiation, motility and survival. Receptor tyrosine kinases are among the primary mediators of signals between the surface of the cell to target proteins in cytoplasmic compartments and in the nucleus. One family of receptor tyrosine kinases, the epidermal growth factor receptors (EGFRs), has been shown to have a critical role in these signal transduction processes.

Members of the epidermal growth factor receptor family (ErbB1/HER1, ErbB2/HER2, ErbB3/HER3, and ErbB4/HER4) are transmembrane tyrosine kinases that are activated by ligand-induced dimerization. (Schreiber et al., (1983) Journal of Biological Chemistry 258(2):846-53; Ushiro and Cohen, (1980) Journal of Biological Chemistry 255(18):8363-5). These receptors regulate cell proliferation, differentiation, and migration, and their abnormal activation is associated with a variety of human cancers. (Yarden and Sliwkowski, (2001) Nature Reviews Molecular Cellular Biology 2(2):127-37). Several cancer drugs (for example, Erlotinib) interact with the ATP-binding site of the EGFR kinase to halt tumor growth and increase apoptosis in cancer cells.

It is known that the EGFR kinase domain is activated after ligand-induced dimerization of the extracellular region of the receptor, although the underlying mechanism has remained elusive. Studies have shown that mutations in the catalytic domain of EGFR can interfere with the kinase activity of these proteins. (Chan et al., (1996) Journal of Biological Chemistry, Vol. 27(37): 22619-23).

The development of compounds that directly inhibit the kinase activity of the EGFR, as well as antibodies that reduce EGFR kinase activity by blocking EGFR activation, are areas of intense research effort (de Bono and Rowinsky, (2002) Trends in Molecular Medicine, Vol. 8 (4 Suppl): S19-26; Dancey and Sausville, (2003) Nature Reviews. Drug Discovery, Vol. 2: 296-313). Several studies have demonstrated or suggested that some EGFR kinase inhibitors might improve tumor cell or neoplasia killing when used in combination with certain other anti-cancer or chemotherapeutic agents or treatments (e.g. Herbst et al., (2002) Expert Opinion on Biological Therapy, Vol. 1(4): 719-32; Solomon et al., (2003) International Journal Radiology, Oncology, Biology, Physics, Vol. 57(1): 713-23; Krishnan et al., (2003) Frontiers in Bioscience, Vol. 8: e1-13; Grunwald and Hidalgo, (2003) Journal of the National Cancer Institute, Vol. 95: 851-67; Seymour, (2003) Current Opinion in Investigational Drugs, Vol. 4(6): 658-66; Khalil et al., (2003) Expert Review on Anticancer Therapy, Vol. 3(3): 367-80; Bulgaru et al., (2003) Expert Review on Anticancer Therapy, Vol. 3(3): 269-79; Ciardiello et al., (2000) Clinical Cancer Research, Vol. 6: 2053-63; and patent Publication No: US 2003/0157104).

The Mig-6 protein has been shown to be a negative modulator of EGFR activity. Ullrich et al (WO 02/067975) described using the protein to inhibit EGFR activity in rat fibroblasts. The interaction between EGFR and Mig-6 was determined using a yeast two hybrid screen. A similar method was used to screen for other potential modulators of EGFR. However, the high rate of false negatives inherent to a yeast two hybrid screen makes such a process inefficient for most drug discovery uses.

Drugs targeting EGFR that are currently in use inhibit EGFR through interaction with the active site, but such pharmaceuticals are not effective for many EGFR-related illnesses.

A need exists, therefore, for methods and compositions for screening for modulators of EGFR.

### SUMMARY OF THE INVENTION

Accordingly, in one aspect, the invention provides a method of targeted drug discovery which includes the steps of: (i) contacting an isolated EGFR kinase domain with a test compound; and (ii) detecting an increase in EGFR kinase domain activity. Such an increase in activity identifies the test compound as an inhibitor of EGFR. In a particularly preferred embodiment, the test compound binds in a hydrophobic pocket between helix C of the EGFR kinase domain and the main body of the EGFR kinase domain

In another aspect, the invention provides a method for screening for potential inhibitors of EGFR activation. This method includes the steps of: (a) attaching an isolated polypeptide corresponding to an EGFR kinase domain to a lipid vesicle surface to form a conjugated polypeptide; (b) determining activity of the conjugated polypeptide; and (c) contacting the conjugated polypeptide with a test compound; (d) comparing the activity of step (b) with the activity of (c). In a preferred embodiment, following step (c), the invention provides a step in which the activity of the conjugated polypeptide is determined. In a still further preferred embodiment, if the activity determined in (c) is less than the activity determined in (b), the comparing step in (d) identifies the test compound as an inhibitor of EGFR activation.

In still another aspect, the invention provides method for inhibiting EGFR activation. This method includes the step contacting an EGFR kinase domain with a test molecule that interacts with said EGFR kinase domain. This contacting between the EGFR kinase domain and the test molecule prevents interaction of the N-lobe of the EGFR kinase domain with the C-lobe of the EGFR kinase domain, thus inhibiting EGFR activation.

Other objects, aspects and advantages of the instant invention are set forth in the detailed description which follows.

### BRIEF DESCRIPTION OF THE DRAWINGS

FIG. 1 shows the sequences of the identified regions of the Mig-6 peptide or the EGFR kinase domain.

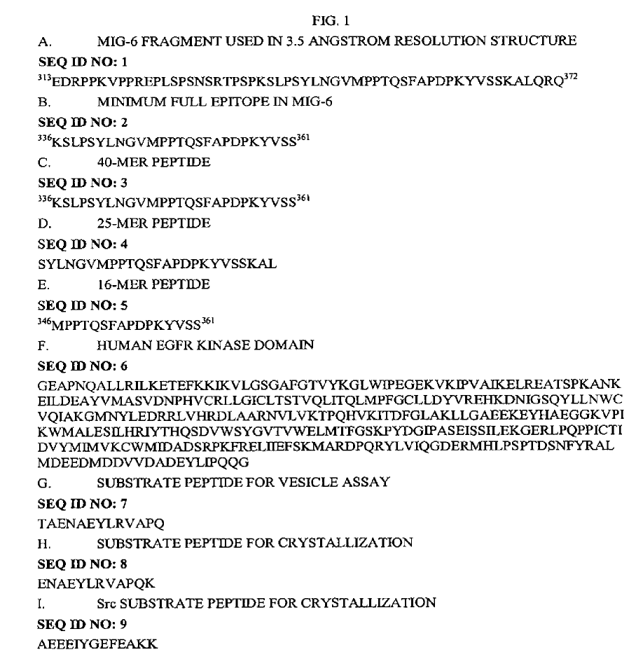

FIG. 2 shows the vector map of the construct used to express the human EGFR kinase domain in Sf9.

FIG. 3 is the nucleotide sequence of the expression vector construct for the EGFR kinase domain.

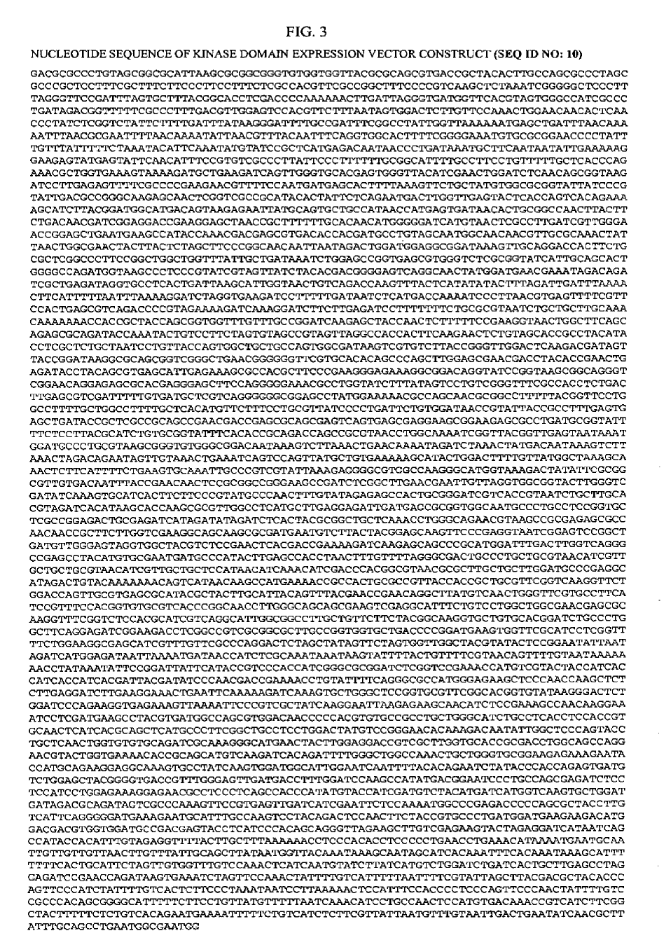

FIG. 4 is a crystal structure of a complex between EGFR kinase domain and the bacterially expressed Mig-6 peptide.

FIG. 5 shows a general view of ligand-induced dimerization and activation of EGFR (A), and a detailed view of the catalytic site of EGFR kinase domain in the active (B) and inactive (C) conformation.

FIG. 6 shows data from a vesicle assay system. FIG. 6A shows catalytic activity of the wildtype and mutant EGFR kinase domains in solution and attached to vesicles. FIG. 6B shows the concentration-dependent activation of the wild-type kinase domain upon attachment to lipid vesicles.

FIG. 7 shows the crystal structure of an EGFR kinase domain in complex with an ATP analog substrate peptide conjugate (A) and in complex with AMP-PNP (B). FIG. 7C shows the crystal structure of an inactive Src kinase in complex with AMP-PNP.

FIG. 8 shows a crystal structure of the asymmetric dimer interface of the EGFR kinase domain. FIG. 8A shows the asymmetric dimer (left panel) in comparison to a CDK2/cyclin A complex (right panel). FIG. 8B shows detailed views of the asymmetric dimer interface.

FIG. 9 displays information regarding the symmetric dimer interface. FIG. 9A shows the residues involved in the symmetric dimer interface. FIG. 9B shows the results of a phosphorylation assay for the wildtype interface and various mutants.

FIG. 10 shows results of a phosphorylation assay of the wildtype dimer and of mutant constructs with mutations in the N-lobe and C-lobe face of the dimer interface.

FIG. 11 is a schematic model of predicted outcomes of various transfection/cotransfection experiments.

FIG. 12 shows the results of a phosphorylation assay of various transfection/cotransfection experiments (left panel) and the effects of mutations in the asymmetric dimer interface on the catalytic activity of the kinase domain in solution and attached to lipid vesicles (right panel).

FIG. 13 is a sequence alignment of EGFR family members from human and mouse. Residues in the N-lobe faces are denoted by ovals, and residues in the C-lobe faces are denoted by triangles. SEQ ID NO: 10.

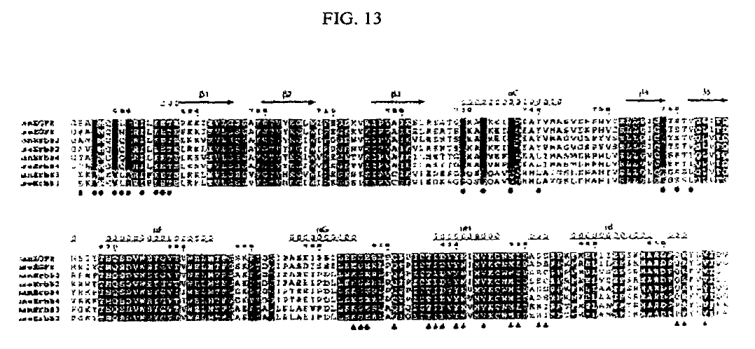

FIG. 14 is a general model of the activation mechanism for the EGFR family receptor tyrosine kinases.

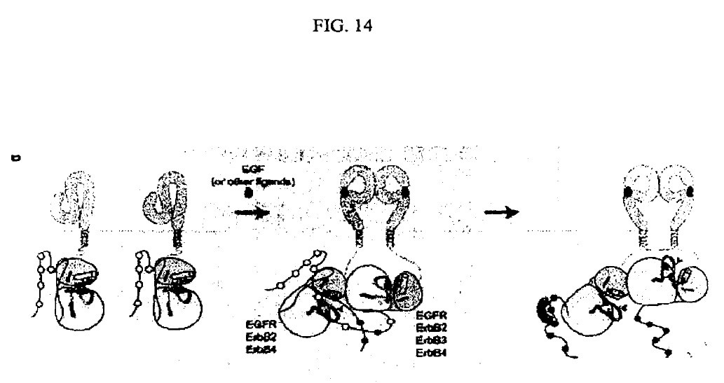

FIG. 15 displays data regarding an EGFR kinase domain monomer. FIG. 15A shows data from an ultracentrifugation experiment of an EGFR kinase domain monomer in solution. The lower panel shows the fit of the data (circles) to a single species ideal model (solid curve), which yielded a molecular weight of 37890 Da. Residuals of the fitting (circles) are plotted in the upper panel. FIG. 15B shows the results of a dynamic light scattering experiment for an EGFR kinase domain monomer in solution.

FIG. 16 shows a representative size distribution of lipid vesicles measured by dynamic light scattering.

FIG. 17 shows higher order oligomers based on the CDK/cyclin-like asymmetric dimer (A) and a comparison of the asymmetric and symmetric dimers (B).

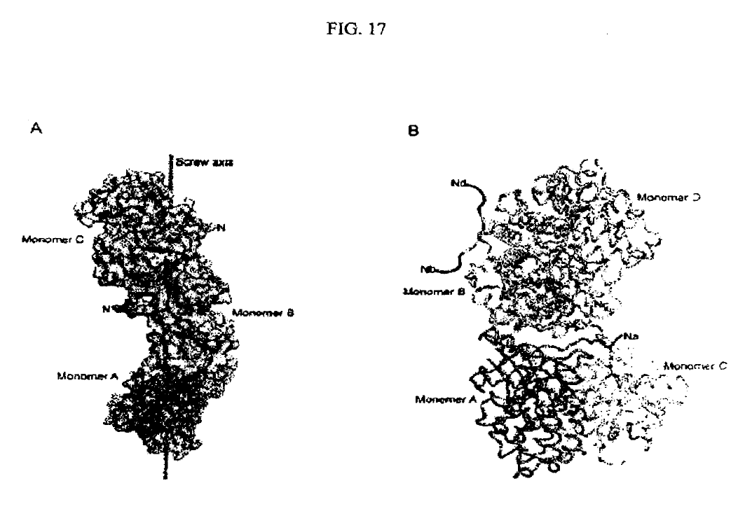

FIG. 18 is a comparison of the active and inactive conformations of the EGFR kinase domain. 18A is a superimposition of the active (ATP analog-peptide conjugate bound) and inactive (AMP-PNP bound V924R mutant) structures. 18B is a superimposition of the structures of the AMP-PNP bound V924R mutant and the Lapatinib-bound wild type EGFR kinase domain.

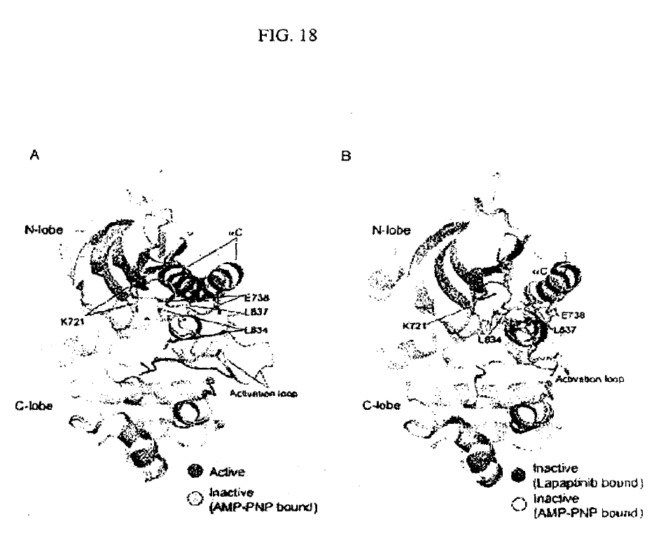

FIG. 19 shows the results of a phosphorylation assay of wildtype and mutant EGFR kinase domains.

FIG. 20 shows data from a mass spectrum analysis of the Y845F mutant EGFR kinase domain.

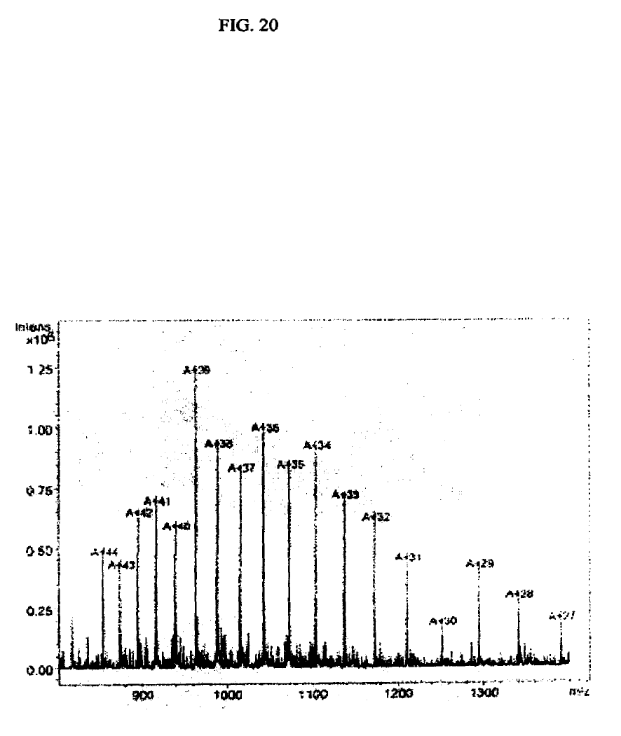

FIG. 21 shows the vector map for the Mig-6 expression vector construct.

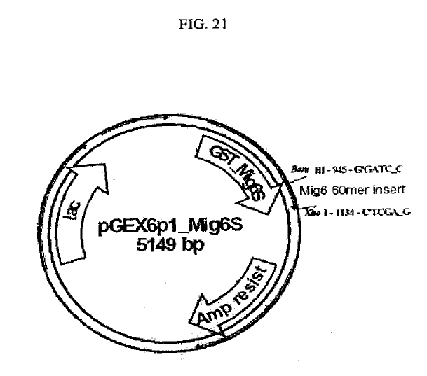

FIG. 22 shows the nucleotide sequence of the Mig-6 expression vector construct. SEQ ID NO: 11.

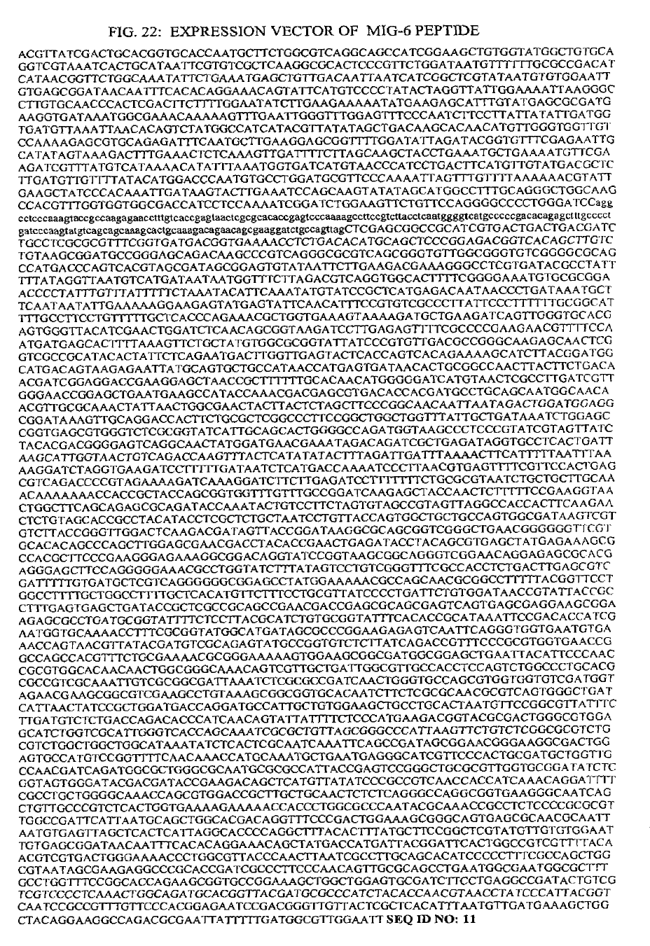

FIG. 23 shows the structure of the EGFR kinase domain/MIG6(segment 1): (a) is a schematic diagram of human MIG6 primary structure; (b) shows to orthogonal view of the EGFR kinase domain/MIG6(segment 1) complex; (c) is a detailed view of the interface between the EGFR kinase domain and MIG6(segment 1); and (d) is a comparison of the MIG6(segment 1) interface and the kinase domain asymmetric dimer interface on the distal surface of the kinase C lobe.

FIG. 24 shows data related to binding and inhibition of EGFR by MIG6(segment 1): (a) shows titrations of the wildtype EGFR kinase domain and the V924R and I682Q mutants to the 30-residue (residues 334-363) fluorescein-labeled MIG6 peptide; (b) shows titrations of the wildtype EGFR kinase domain to the wildtype and three mutant 30-residue fluorescein-labeled peptides; (c) shows inhibition of the activity of the EGFR kinase domain by peptides spanning MIG6(segment 1) in the vesicle-based kinase assay; (d) shows a cell-based assay of MIG6 and segment 1 on full-length EGFR auto-phosphorylation.

FIG. 25 shows data related to inhibition of EGFR kinase activity by MIG6(segments 1-2): (a) shows inhibition of the L834R mutant kinase in solution by peptides 336-412 or 336-412(Y358A); the insert shows an expanded view at low peptide concentrations; and (b) shows inhibition of the wildtype kinase in solution by peptides 336-412 or 336-412(Y358A).

FIG. 26 shows data and schematic diagrams related to a mechanism for EGFR inhibition by MIG6: (a) shows data from a co-transfection experiment in which activation of EGFR(activatable) can be activated by EGFR(activator), and this activation can be inhibited by MIG6; the cartoon underneath the gel data illustrates the co-transfection combinations; (b) shows data from a co-transfection experiment in which full-length EGFR with a L834R/V924R double mutation is activated only when co-transfected with EGFR(activator); the cartoon underneath the gel data illustrates the co-transfection combinations; and (c) is a schematic diagram showing the double-headed mechanism for EGFR inhibition by MIG6 involving both segment 1 and segment 2.

### DETAILED DESCRIPTION OF THE INVENTION

### Introduction

The present invention relates to screening for compounds which inhibit, regulate and/or modulate epidermal growth factor receptor (EGFR) activity, as well as compositions which contain these compounds. The invention also provides methods of using the compounds of the instant invention to treat EGFR-activation-dependent diseases and conditions, such as angiogenesis, cancer, tumor growth, atherosclerosis, age related macular degeneration, diabetic retinopathy, and inflammatory diseases.

### DEFINITIONS

“EGFR” refers to Epidermal Growth Factor Receptor. All EGFR family members are encompassed by the present invention. As used herein unless otherwise identified, the term “EGFR” refers to any receptor protein tyrosine kinase belonging to the ErbB receptor family, including without limitation HER1, HER2, HER3, HER4, as well as any other members of this family to be identified in the future. The EGFR receptor will generally comprise an extracellular domain, which may bind an EGFR ligand; a lipophilic transmembrane domain; a conserved intracellular tyrosine kinase domain; and a carboxyl-terminal signaling domain harboring several tyrosine residues which can be phosphorylated. EGFR may be a “native sequence” EGFR or an “amino acid sequence variant” thereof.

A “native sequence” is a sequence of amino acid residues as it is found in nature, without modification by artificial means.

An “amino acid sequence variant” is a naturally occurring or artificially mutated or altered version of a native amino acid sequence.

“EGFR” includes naturally occurring mutant forms, e.g., additions, substitutions and deletions, as well as recombinant forms generated using molecular biology techniques.

An “EGFR molecule” encompasses the amino acid sequence encoding for EGFR. The term also encompasses less than complete fragments of the amino acid sequence, as well as proteins, polypeptides and polypeptide fragments derived from a full-length EGFR protein.

An “EGFR encoding nucleic acid” encompasses the nucleotide sequence encoding for EGFR. The term also encompasses less than full-length nucleotide sequences, as well sequences which have been altered, e.g., mutated with insertions, deletions, and substitutions, and sequences which have been inserted into delivery vehicles, such as recombinant expression vectors.

The “activity” of a polypeptide or protein refers to a functional property associated with that molecule. For example, “EGFR activity” can refer to the tyrosine kinase activity of the molecule as well as the process of dimerization upon binding a ligand. The specific activity associated with a polypeptide or protein can also be identified through a description of a functional process, e.g., phosphorylation.

The terms “EGFR protein” and “EGFR polypeptide” are used interchangeably and encompass full length, wildtype, fragment, variant and mutant EGFR molecules. The terms encompass polypeptides having an amino acid sequence which substantially corresponds to at least one 10 to 50 residue (e.g., 10, 20, 25, 30, 35, 40, 45, 50) amino acid fragment and/or a sequence homologous to a known EGFR or group of EGFRs, wherein the EGFR polypeptide has homology of at least 80%, such as at least 81, 82, 83, 84, 85, 86, 87, 88, 89, 90, 91, 92, 93, 94, 95, 96, 97, 98, 99 or 100% homology, to the sequence of said known EGFR or group of EGFRs, and exhibits EGFR activity. Encompassed in the present invention is an EGFR polypeptide which is not naturally occurring or is naturally occurring but is in a purified or isolated form which does not occur in nature.

An amino acid or nucleic acid is “homologous” to another if there is some degree of sequence identity between the two. Preferably, a homologous sequence will have at least about 85% sequence identity to the reference sequence, preferably with at least about 90% to 100% sequence identity, more preferably with at least about 91% sequence identity, with at least about 92% sequence identity, with at least about 93% sequence identity, with at least about 94% sequence identity, more preferably still with at least about 95% to 99% sequence identity, preferably with at least about 96% sequence identity, with at least about 97% sequence identity, with at least about 98% sequence identity, still more preferably with at least about 99% sequence identity, and about 100% sequence identity to the reference amino acid or nucleotide sequence.

A “kinase domain” is a region of a polypeptide or protein that shows kinase activity. A kinase domain may be defined in structural terms with reference to an amino acid sequence or to a crystallographic structure.

“EGFR kinase domain molecule” encompasses amino acid sequences corresponding to an EGFR kinase domain. The EGFR kinase domain is a tyrosine kinase domain and in the wildtype human protein is located from amino acid residues 672 to 998. The terms “EGFR kinase domain” and “EGFR kinase domain molecule” are interchangeable and encompass the full wildtype domain, fragments of the domain, as well as mutants and variations of the domain.

A “dimer” is a molecule that comprises two simpler, often identical molecules. When both components (also called “subunits”) of a dimer are identical to each other, the dimer can also be referred to as a “homodimer”, while a dimer comprising non-identical subunits can be referred to as a “heterodimer”. An “EGFR dimer” is a dimer in which at least one subunit corresponds to a member of the ErbB receptor family. “EGFR dimer”, “EGFR molecule” and “EGFR protein” can be used interchangeably.

“Dimer formation” encompasses the joining of two subunits to form a dimer. Dimer formation can occur between full-length proteins as well as polypeptides corresponding to a specific epitope or domain of a protein, such as a kinase domain of an EGFR molecule. “Dimer formation” and “dimerization” can be used interchangeably and encompass the activation of an EGFR molecule as well as the coming together and joining of two subunits of an EGFR molecule.

An “asymmetric dimer interface” refers to the region of an EGFR dimer in which the C-lobe of a kinase domain of one subunit is juxtaposed against the N-lobe of a kinase domain of the other subunit.

The term “mutant EGFR” encompasses naturally occurring mutants and mutants created chemically and/or using recombinant DNA techniques. “Mutant EGFR” and “mutant EGFR molecules” can be used interchangeably.

“C-terminal lobe” and “C-lobe” can be used interchangeably and refer to the C-terminal region of an EGFR monomer composed mainly of helical domains (see, e.g. Zhang et al., Cell 125 1137-1149 Jun. 15, 2006).

The term “distal” refers to a location that is a distance away from a reference point. Thus, a residue located “distal from the catalytic domain” is a residue located outside of the defined catalytic domain.

“Modulation” of a protein encompasses changes to either the structure of a protein or to the functional activity of a protein.

A “vesicle assay system” comprises vesicles used to measure a functional activity of a molecule. An exemplary “vesicle” is a closed shell, generally derived from a lipid (e.g., a membrane) by a physiological process or through mechanical means. Preferably, a vesicle comprises one or more types of lipids and has a diameter from about 100 nm to about 200 nm.

“Localizing” and “to localize” (as in “localizing a kinase domain molecule to surface of lipid vesicle”) refers to a process of delivering an entity to a specified location, wherein that location is described generally (e.g. “a surface”) or specifically (e.g. “to amino acid residue 273”).

To be “conjugated” refers to the process or characteristic of being joined. For example, a protein conjugated to a lipid vesicle is joined to that vesicle by means of some kind of interaction, such as a covalent or hydrophobic bond.

A “therapeutic” is a drug or pharmaceutical composition provided to prevent, to alleviate the symptoms of or to cure an illness or disease. An “effective” therapeutic is one which is able to create these effects at a particular concentration.

A “functional assay” is an assay of a functional property of a molecule. For example, a functional assay of a tyrosine kinase may measure the level of phosphorylation upon application of that molecule to a sample. Similarly, “functional effects” refers to changes in a molecule or an action upon a molecule that somehow changes the functional properties of that molecule.

A “tag molecule” (e.g., a “histidine tag”) is a molecule added to another molecule to act as an identifier or to modulate a certain property of the attached molecule, such as the ability to bind to yet another molecule. Tag molecules can also be used in methods for purifying or immobilizing the attached molecules.

The “catalytic activity” of a molecule, particularly a protein, refers to the ability of that molecule to increase the rate of a reaction without becoming consumed.

A “hexa-histidine tag” is an epitope tag comprising six histidine amino acid residues in sequence that can serve as a tag without affecting functional properties of the protein to which it is attached.

The term “structural analysis” encompasses techniques used to model the three-dimensional features of a protein, including without limitation X-ray crystallography, computer modeling predictions based on amino acid sequence, and biochemical analysis of protein domain interaction.

“Mig-6”, “Mig-6 polypeptide” “Mig-6 protein” can be used interchangeably and encompass the molecule (also known as Gene 33 and RALT) which is known to negatively regulate EGFR activity. Mutation of Mig-6 expression is implicated in EGFR activation-associated cancers (Anastasi et al., 2003; Ferby et al., 2006, Zhang et al., 2006). These terms also encompass fragments of Mig-6.

An “isolated” molecule, such as an isolated polypeptide or isolated nucleic acid, is one which has been identified and separated and/or recovered from a component of its natural environment. The identification, separation and/or recovery are accomplished through techniques known in the art, or readily available modifications thereof.

An “allosteric” mechanism refers to a mechanism of action in which a molecule combines with a site on the protein other than the active site. In an exemplary embodiment, the combination results in a change in the protein's conformation, e.g., at or proximate to the active site.

The term “therapeutically effective amount” refers to an amount of a drug effective to treat, cure, prevent or ameliorate a disease or disorder in a mammal. In the case of cancer, the therapeutically effective amount of the drug may reduce the number of cancer cells; reduce the tumor size, inhibit (i.e., slow to some extent and preferably stop) cancer cell infiltration into peripheral organs, inhibit (i.e., slow to some extent and preferably stop) tumor metastasis, inhibit, to some extent, tumor growth, and/or relieve to some extent one or more of the symptoms associated with the cancer.

“Polypeptide” refers to a polymer in which the monomers are amino acids and are joined together through amide bonds, alternatively referred to as a peptide. When the amino acids are α-amino acids, either the L-optical isomer or the D-optical isomer can be used. Additionally, unnatural amino acids, for example, β-alanine, phenylglycine and homoarginine are also included. Commonly encountered amino acids that are not gene-encoded may also be used in the present invention. All of the amino acids used in the present invention may be either the D- or L-isomer. The L-isomers are generally preferred. In addition, other peptidomimetics are also useful in the present invention. For a general review, see, Spatola, A. F., in CHEMISTRY AND BIOCHEMISTRY OF AMINO ACIDS, PEPTIDES AND PROTEINS, B. Weinstein, eds., Marcel Dekker, New York, p. 267 (1983).

As used herein, “amino acid” refers to a group of water-soluble compounds that possess both a carboxyl and an amino group attached to the same carbon atom. Amino acids can be represented by the general formula NH₂—CHR—COOH where R may be hydrogen or an organic group, which may be nonpolar, basic acidic, or polar. As used herein, “amino acid” refers to both the amino acid radical and the non-radical free amino acid.

The terms “cancer” and “cancerous” refer to or describe the physiological condition in mammals that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

A cancer “characterized by excessive activation” of EGFR is one in which the extent of EGFR activation in cancer cells significantly exceeds the level of activation of that receptor in non-cancerous cells of the same tissue type. Such excessive activation may result from overexpression of EGFR and/or greater than normal levels of an EGFR ligand available for activating the EGFR receptor in the cancer cells. Overexpression of EGFR may refer to greater than normal levels of EGFR protein or mRNA. Excessive activation of EGFR may cause and/or be caused by the malignant state of a cancer cell.

### Inhibition of EGFR

In one aspect, the present invention provides compositions and method for the modulation of EGFR activation.

In another aspect, the invention provides novel inhibitors of EGFR. In a further aspect, the invention provides inhibitors which act by preventing activation of EGFR. In a still further aspect, the inhibitors prevent formation of an asymmetric dimer interface between EGFR monomers. In such a mechanism of inhibition, the EGFR molecule retains a basal level of activity but is inhibited from activating, i.e. is prevented from prompting the signal transduction cascade that would normally develop upon binding of a ligand to the extracellular activation loop of EGFR (also referred to herein as the “ligand binding region of EGFR”). In one embodiment, the present invention provides inhibitors which bind to the kinase domain of the EGFR molecule, thereby preventing formation of the asymmetric dimer interface, which in turn prevents activation of EGFR.

In a preferred aspect, the invention provides compositions for the inhibition of EGFR, wherein those compositions comprise molecules which prevent formation of an asymmetric dimer interface between EGFR monomers. Such molecules include polypeptides, small molecules, peptidomimetics, and other molecules and compositions which are able to prevent formation of the asymmetric dimer interface. In a further embodiment, the inhibitors of the invention comprise isolated polypeptides. In a still further embodiment, the isolated polypeptides comprise the Mig-6 protein and/or fragments of Mig-6, as is discussed more fully below.

In a preferred aspect, the invention provides a pharmaceutical composition comprising one or more isolated polypeptides with an amino acid sequence selected from SEQ ID NOs: 1-9, wherein said one or more polypeptides are combined with at least one pharmaceutically acceptable carrier. In one embodiment, the isolated polypeptides are inhibitors of EGFR. In a further embodiment, the pharmaceutical composition is administered to patients diagnosed with illnesses associated with EGFR. Administration of such a pharmaceutical composition is accomplished using techniques known in the art and those described herein.

Mig-6

Mig-6, which is also identified as Gene 33 and RALT, is known to negatively regulate EGFR activity and mutation or loss of Mig-6 expression is implicated in EGFR activation-associated cancers. There is evidence to suggest that Mig-6 inhibits EGFR via an allosteric mechanism. (Zhang et al., (2006) Cell, Vol. 125: 1137-49). The present invention thus provides novel inhibitors of EGFR activation which are derived from the Mig-6 protein.

In a preferred aspect of the invention, Mig-6, or fragments of Mig-6, are expressed in and purified from E. coli. A minimum epitope for EGFR binding has a sequence which comprises SEQ ID NO: 2. In one embodiment, the invention provides an allosteric inhibitor of EGFR activation, where the inhibitor is an isolated polypeptide comprising an amino acid sequence selected from SEQ ID NOs 1-9.

In another aspect of the invention, a 25-mer peptide corresponding to residues 340-364 in Mig-6 (SEQ ID NO: 4) is synthesized. Such a peptide can inhibit activated EGFR kinase at an IC50 of ˜100 μM, suggesting that the 25-mer peptide does not comprise the entire binding epitope. A crystal structure of the 25-mer peptide crystallized with the EGFR kinase domain identifies the region of the peptide bound to the kinase as containing 16 residues: MPPTQSFAPDPKYVSS.

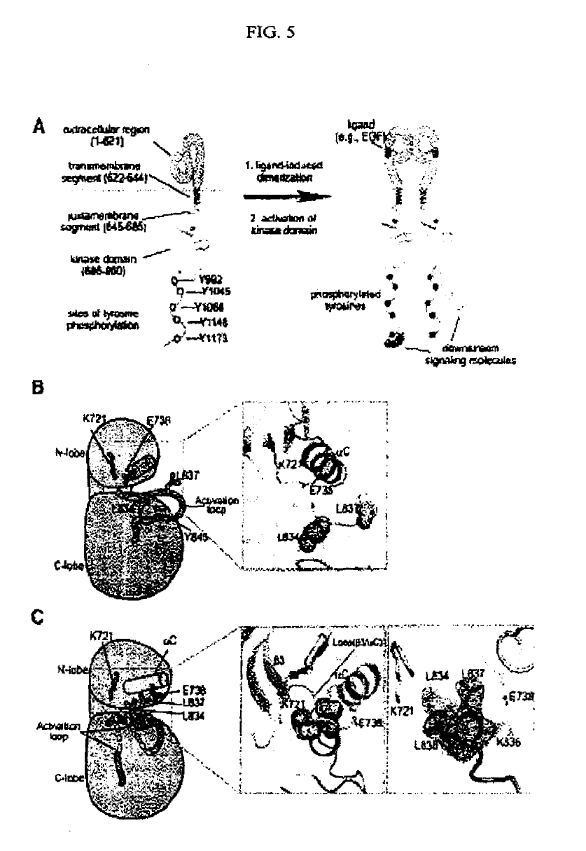

In another aspect of the invention, a 40-mer peptide comprising amino acid sequence SEQ ID NO: 3 is synthesized. The 40-mer peptide is much more potent than the 25-mer peptide in inhibiting the activated EGFR kinase, with an IC50˜10 μM. A crystal structure of the complex of the EGFR kinase domain and the 40-mer peptide has improved resolution (˜2.9 Å) and can be used, similar to the description above for the 25-mer peptide, to identify residues of interaction between the peptide and the kinase domain. (FIG. 5).

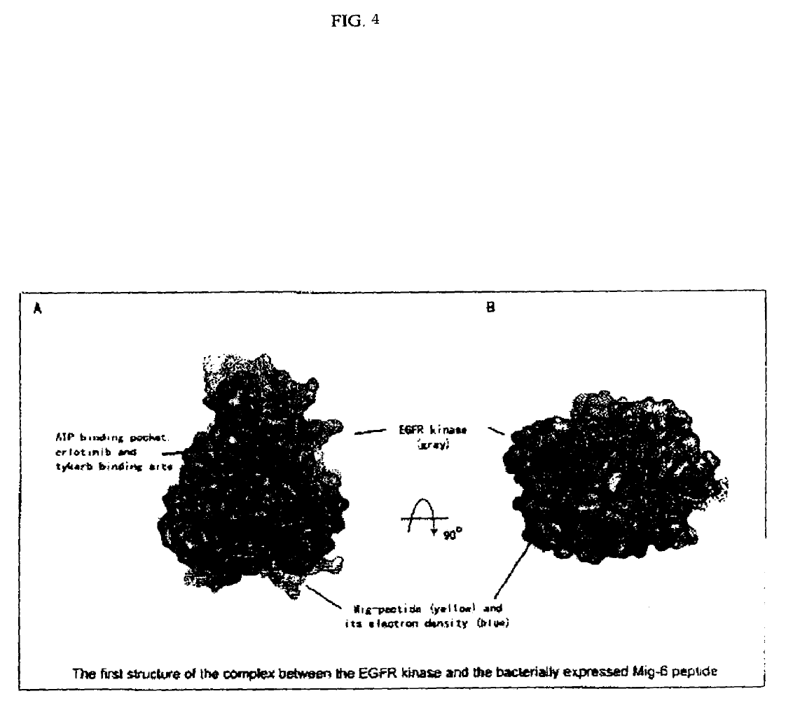

The Mig-6 peptide binds the EGFR kinase domain by wrapping around a shallow groove on the surface of the base of the kinase domain (FIG. 4). At this face of the kinase domain, a number of conserved nonpolar residues form a hydrophobic surface which interacts specifically with the N-lobe of the other kinase upon the formation of the asymmetric activating kinase dimer. Several hydrophobic residues in the Mig-6 peptide pack tightly against this hydrophobic surface in the C-lobe of the kinase, preventing the formation of the asymmetric dimer and thus inhibiting EGFR kinase activation.

In one aspect of the invention, the binding affinity of a peptide to the EGFR kinase domain is improved by modifying the peptide sequence to more tightly interact with the hydrophobic surface in the C-lobe of the kinase domain. In one embodiment, the peptide sequence is modified with reference to the residues of interaction between the EGFR kinase domain and a Mig-6 polypeptide comprising an amino acid sequence comprising SEQ ID NOs: 1-5.

In another aspect of the invention, small molecule mimics of the Mig-6 peptide are designed which bind to the kinase at the same structural features shown in the crystal structures. Such peptides and small molecules can be developed into new classes of EGFR-antagonizing drugs for cancer therapy in accordance with the present invention.

Mig-6 and EGFR kinase domains are expressed and purified according to techniques known in the art and as described herein (see Example I).

In another aspect, the invention provides a method of treatment for cancer, where the treatment involves (1) determining the types of EGFR molecules expressed in tumor cells associated with the cancer, and (2) administering one or more inhibitors that are able to interact with the types of EGFR molecules identified in step (1). In one embodiment, the inhibitors are peptides, peptidomimetics, small molecules, and other molecules and compositions which are able to prevent formation of the asymmetric dimer interface between EGFR monomers. In a preferred embodiment, the EGFR inhibitors are isolated polypeptides which are able to bind to the kinase domain of the identified EGFR molecules, thereby preventing formation of the asymmetric dimer interface. In a further embodiment, the isolated polypeptides comprise D-, L-, and unnatural isomers of amino acids. In a still further embodiment, the isolated polypeptides have at least 70% sequence identity to SEQ ID NOs: 1-9.

In a further aspect, methods for treating cancer with EGFR inhibitors are provided, wherein the treatment prevents the excessive or uncontrolled cell growth that can lead to the development of tumors. Tumors suitable for treatment within the context of this invention include, but are not limited to, breast tumors, gliomas, melanomas, prostate cancer, hepatomas, sarcomas, lymphomas, leukemias, ovarian tumors, thymomas, nephromas, pancreatic cancer, colon cancer, head and neck cancer, stomach cancer, lung cancer, mesotheliomas, myeloma, neuroblastoma, retinoblastoma, cervical cancer, uterine cancer, and squamous cell carcinoma of skin. Many known cell surface receptors are generally preferentially expressed in tumors, and ligands for these receptors can be used to inhibit the progression and development of tumor cells. Such ligands can include known ligands for the receptors, molecules and compounds that are identified using methods of the invention as being able to interact with such receptors, as well as ligands specifically designed and developed for particular receptors—such as by raising antibodies to the receptors and by designing novel molecules with structures that allow interaction with particular receptors.

Through delivery of the compositions of the present invention, unwanted growth of cells may be slowed or halted, thus ameliorating the disease. This treatment is suitable for warm-blooded animals: mammals, including, but not limited to, humans, horses, dogs, and cats, and for non-mammals, such as avian species. Methods of treating such animals with compositions of the present invention are provided herein.

### EGFR and Disease

The compounds of the present invention are in one aspect provided for the treatment of disorders in which aberrant expression ligand/receptor interactions or activation or signaling events related to EGFR are involved. Such disorders may include those of neuronal, glial, astrocytal, hypothalamic, and other glandular, macrophagal, epithelial, stromal, and blastocoelic nature in which aberrant function, expression, activation or signaling of EGFR is involved. In an additional aspect, the compounds of the present invention may have therapeutic utility in inflammatory, angiogenic and immunologic disorders involving both identified and as yet unidentified EGFRs and other tyrosine kinases that are inhibited by the compounds of the present invention.

In one aspect, the invention provides a method for the treatment of abnormal cell growth in a mammal which comprises administering to said mammal an amount of a compound or composition, or a pharmaceutically acceptable salt, solvate or prodrug thereof, that is effective in treating abnormal cell growth. This treatment can in an exemplary embodiment be administered in combination with another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens. In one embodiment, the invention provides a pharmaceutical composition for treating abnormal cell growth wherein the composition includes a compound which inhibits EGFR activation, or a pharmaceutically acceptable salt, solvate or prodrug thereof, that is effective in treating abnormal cell growth, and another anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, antibodies, cytotoxics, anti-hormones, and anti-androgens.

EGFR is frequently overexpressed in cancer. (Mendelsohn et al., (2006) Semin Oncol. 33(4):369-85). Arthritis, hypersecretory respiratory diseases, and skin conditions such as psoriasis are also associated with EGFR overexpression and activation. Accordingly, a preferred aspect of the instant invention provides methods and compositions for the inhibition of EGFR, wherein said inhibition serves as a treatment for EGFR-associated diseases such as cancer and arthritis. In a particularly preferred embodiment, the invention provides methods and compositions for the inhibition of EGFR in which said methods and compositions prevent the formation of an asymmetric dimer interface.

With regard to cancer, two of the major hypotheses advanced to explain the excessive cellular proliferation that drives tumor development relate to functions known to be kinase regulated. That is, it has been suggested that malignant cell growth results from a breakdown in the mechanisms that control cell division and/or differentiation. It has been shown that the protein products of a number of proto-oncogenes are involved in the signal transduction pathways that regulate cell growth and differentiation. These protein products of proto-oncogenes include growth factor receptors such as EGFR. It is thus a preferred aspect of the present invention to provide a cancer treatment in which a composition of the invention that is able to prevent the cell division and/or differentiation processes that lead to malignant cell growth of cancer. Such a cancer treatment, in a preferred embodiment, halts or slows down cell division and/or differentiation by preventing formation of the EGFR asymmetric dimer interface, thereby preventing the intracellular second messenger cascade that takes place upon activation of an EGFR dimer by intermolecular interaction or by activation upon binding of an extracellular ligand.

For patients with lung cancer, the EGFR inhibitor Erlotinib increases survival times by several months (Bezjak et al., (2006) Journal of Clinical Oncology, Vol. 24(24): 3831-7). In vitro studies have shown that another EGFR inhibitor, the drug gefitinib (marketed as Iressa), is able to halt the growth of cancer cells in colon cancer (Azzariti et al., (2006) World Journal of Gastroenterol, Vol. 12(32): 5140-7 Wiedmann et al., (2006) Anticancer Drugs, Vol. 17(7): 783-95), and biliary tract cancer (Wiedmann et al., (2006)Anticancer Drugs, Vol. 17(7): 783-95). Gefitinib has also been shown to increase apoptosis of gastric cancer cells (Rojo et al., (2006) Journal of Clinical Oncology, Vol. 24(26): 4309-16). Erlotinib and gefitinib have both been shown to be effective as part of combination therapies, in which the synergistic effects of the EGFR inhibitors combined with radiotherapy significantly improved outcomes over those seen with radiotherapy alone (Park et al., (2006) Cancer Research, Vol. 66(17): 8511-19). Lapatinib, another EGFR inhibitor, is currently in Phase III clinical trials for treatment of breast cancer (Johnston et al., (2006) Drugs of Today, Vol. 42(7): 441-53). Studies have also shown that EGFR inhibitors can be used to treat, ameliorate and prevent illnesses not associated with cancer. For example, EGFR inhibitors have been shown to prevent parathyroid hyperplasia, which is the cause of parathyroid gland enlargement in kidney disease (Dusso et al., (2006) Kidney International Supplement, Vol. 102: S8-11).

Other pathogenic conditions which have been associated with tyrosine kinases such as EGFR include, without limitation, psoriasis, hepatic cirrhosis, diabetes, angiogenesis, restenosis, ocular diseases, rheumatoid arthritis and other inflammatory disorders, immunological disorders such as autoimmune disease, cardiovascular disease such as atherosclerosis and a variety of renal disorders. Thus, in a preferred aspect of the invention, compositions and methods are provided for the treatment of these EGFR-associated diseases, in which one exemplary embodiment of the invention treats, prevents, ameliorates, or cures the disease by preventing uncontrolled cell differentiation and proliferation.

In another aspect of the invention, compositions and methods are provided for the treatment, amelioration, and prevention of angiogenesis-dependent diseases. In these diseases, vascular growth is excessive or allows unwanted growth of other tissues by providing blood supply. These diseases include angiofibroma, arteriovenous malformations, arthritis, atherosclerotic plaques, corneal graft neovascularization, delayed wound healing, diabetic retinopathy, granulations due to bums, hemangiomas, hemophilic joints, hypertrophic scars, neovascular glaucoma, nonunion fractures, Osler-weber syndrome, psoriasis, pyogenic granuloma, retrolental fibroplasia, scleroderma, solid tumors, trachoma, and vascular adhesions.

By inhibiting vessel formation (angiogenesis), unwanted growth may be slowed or halted, thus ameliorating the disease. In a normal vessel, a single layer of endothelial cells lines the lumen. Growth of a vessel requires proliferation of endothelial cells and smooth muscle cells, which is often dependent on EGFR activation. As such, the present invention provides compositions and methods for the inhibition of EGFR activation.

In a further embodiment, the present invention provides compounds for the chemoprevention of cancer. Chemoprevention is defined as inhibiting the development of invasive cancer by either blocking the initiating mutagenic event or by blocking the progression of pre-malignant cells that have already suffered an insult or inhibiting tumor relapse. Chemoprevention may be accomplished in accordance with the present invention by administering compositions described herein to a patient using methods and techniques known in the art and as described herein. In a still further embodiment, chemoprevention is accomplished using the compositions of the present invention alone, in a pharmaceutical formulation or salt, and in combination with one or more other anti-cancer and/or anti-tumor agents.

Formulations and Administration

The compositions of the present invention may in an exemplary embodiment be formulated into preparations in solid, semi-solid, liquid or gaseous forms such as tablets, capsules, powders, granules, ointments, solutions, depositories, inhalants and injections, and usual ways for oral, parenteral or surgical administration. The invention also embraces pharmaceutical compositions which are formulated for local administration, such as by implants.

A compound of the present invention or a physiologically acceptable salt thereof, can be administered as such to a human patient or can be administered in pharmaceutical compositions in which the foregoing materials are mixed with suitable carriers or excipient(s). Techniques for formulation and administration of drugs may be found in “Remington's Pharmacological Sciences,” Mack Publishing Co., Easton, Pa., latest edition.

As used herein, “administer” or “administration” refers to the delivery of a compound or salt of the present invention or of a pharmaceutical composition containing a compound or salt of this invention to an organism for the purpose of prevention or treatment of an EGFR-related disorder.

Suitable routes of administration may include, in an exemplary embodiment without limitation, oral, rectal, transmucosal or intestinal administration or intramuscular, subcutaneous, intramedullary, intrathecal, direct intraventricular, intravenous, intravitreal, intraperitoneal, intranasal, or intraocular injections. The preferred routes of administration are oral and parenteral.

Alternatively, one may administer the compound in a local rather than systemic manner, for example, via injection of the compound directly into a solid tumor, often in a depot or sustained release formulation.

Furthermore, one may administer the drug in a targeted drug delivery system, for example, in a liposome coated with tumor-specific antibody. The liposomes will be targeted to and taken up selectively by the tumor.

Pharmaceutical compositions of the present invention may be manufactured by processes well known in the art, e.g., by means of conventional mixing, dissolving, granulating, dragee-making, levigating, emulsifying, encapsulating, entrapping, lyophilizing processes or spray drying.

Pharmaceutical compositions for use in accordance with the present invention may be formulated in conventional manner using one or more physiologically acceptable carriers comprising excipients and auxiliaries which facilitate processing of the active compounds into preparations which can be used pharmaceutically. Proper formulation is dependent upon the route of administration chosen.

For injection, the compounds of the invention may be formulated in aqueous solutions, preferably in physiologically compatible buffers such buffers with or without a low concentration of surfactant or co-solvent, or physiological saline buffer. For transmucosal administration, penetrants appropriate to the barrier to be permeated are used in the formulation. Such penetrants are generally known in the art.

For oral administration, the compounds can be formulated by combining the active compounds with pharmaceutically acceptable carriers well known in the art. Such carriers enable the compounds of the invention to be formulated as tablets, pills, lozenges, dragees, capsules, liquids, gels, syrups, slurries, suspensions and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores.

Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethyl-cellulose, sodium carboxymethylcellulose, and/or polyvinyl- pyrrolidone (PVP). If desired, disintegrating agents may be added, such as cross-linked polyvinyl pyrrolidone, agar, or alginic acid. A salt such as sodium alginate may also be used.

In one embodiment, the invention provides dragee cores with suitable coatings. For this purpose, concentrated sugar solutions may be used which may optionally contain gum arabic, talc, polyvinyl pyrrolidone, carbopol gel, polyethylene glycol, titanium dioxide, lacquer solutions, and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin, as well as soft, sealed capsules made of gelatin and a plasticizer, such as glycerol or sorbitol. The push-fit capsules can contain the active ingredients in admixture with a filler such as lactose, a binder such as starch, and/or a lubricant such as talc or magnesium stearate and, optionally, stabilizers. In soft capsules, the active compounds may be dissolved or suspended in suitable liquids, such as fatty oils, liquid paraffin, liquid polyethylene glycols, cremophor, capmul, medium or long chain mono- di- or triglycerides. Stabilizers may be added in these formulations, also.

For administration by inhalation, compounds for use according to the present invention may in an exemplary embodiment be conveniently delivered in the form of an aerosol spray using a pressurized pack or a nebulizer and a suitable propellant, e.g., without limitation, dichlorodifluoromethane, trichlorofluoromethane, dichlorotetra-fluoroethane or carbon dioxide. In the case of a pressurized aerosol, the dosage unit may be controlled by providing a valve to deliver a metered amount. Capsules and cartridges of, for example, gelatin for use in an inhaler or insufflator may be formulated containing a powder mix of the compound and a suitable powder base such as lactose or starch.

The compounds may also be formulated for parenteral administration, e.g. by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating materials such as suspending, stabilizing and/or dispersing agents.

Pharmaceutical compositions for parenteral administration include aqueous solutions of a water soluble form, such as, without limitation, a salt, of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances which increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions.

Alternatively, the active ingredient may be in powder form for constitution with a suitable vehicle, e.g., sterile, pyrogen-free water with or without additional surfactants or cosolvents such as POLYSORBATE 80, Cremophor, cyclodextrin sulfobutylethyl, propylene glycol, or polyethylene glycol e.g., PEG-300 or PEG 400, before use.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Alternatively, other delivery systems for hydrophobic pharmaceutical compounds may be employed. Liposomes and emulsions are well known examples of delivery vehicles or carriers for hydrophobic drugs. In addition, certain organic solvents such as dimethylsulfoxide also may be employed, although often at the cost of greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions herein also may comprise suitable solid or gel phase carriers or excipients. Examples of such carriers or excipients include, but are not limited to, calcium carbonate, calcium phosphate, various sugars, starches, cellulose derivatives, gelatin, and polymers such as polyethylene glycols.

Many of the EGFR modulating compounds of the invention may be provided as physiologically acceptable salts wherein the claimed compound may form the negatively or the positively charged species. Examples of salts in which the compound forms the positively charged moiety include, without limitation, quaternary ammonium (defined elsewhere herein), salts such as the hydrochloride, sulfate, citrate, mesylate, lactate, tartrate, maleate, succinate wherein the nitrogen atom of the quaternary ammonium group is a nitrogen of the selected compound of this invention which has reacted with the appropriate acid. Salts in which a compound of this invention forms the negatively charged species include, without limitation, the sodium, potassium, calcium and magnesium salts formed by the reaction of a carboxylic acid group in the compound with an appropriate base (e.g. sodium hydroxide (NaOH), potassium hydroxide (KOH), Calcium hydroxide (Ca(OH)₂), etc).

It is also an aspect of this invention that a compound described herein, or its salt, is combined with other chemotherapeutic agents for the treatment of the diseases and disorders discussed above. In an exemplary embodiment, a compound or salt of this invention is combined with alkylating agents such as fluorouracil (5-FU) alone or in further combination with leukovorin; or other alkylating agents such as, without limitation, other pyrimidine analogs such as UFT, capecitabine, gemcitabine and cytarabine, the alkyl sulfonates, e.g., busulfan (used in the treatment of chronic granulocytic leukemia), improsulfan and piposulfan; aziridines, e.g., benzodepa, carboquone, meturedepa and uredepa; ethyleneimines and methylmelamines, e.g., altretamine, triethylenemelamine, triethylenephosphoramide, triethylenethiophosphoramide and trimethylolmelamine; and the nitrogen mustards, e.g., chlorambucil (used in the treatment of chronic lymphocytic leukemia, primary macroglobulinemia and non-Hodgkin's lymphoma), cyclophosphamide (used in the treatment of Hodgkin's disease, multiple myeloma, neuroblastoma, breast cancer, ovarian cancer, lung cancer, Wilm's tumor and rhabdomyosarcoma), estramustine, ifosfamide, novembrichin, prednimustine and uracil mustard (used in the treatment of primary thrombocytosis, non-Hodgkin's lymphoma, Hodgkin's disease and ovarian cancer); and triazines, e.g., dacarbazine (used in the treatment of soft tissue sarcoma).

In a further embodiment, a compound or salt of this invention is provided in combination with other antimetabolite chemotherapeutic agents such as, without limitation, folic acid analogs, e.g. methotrexate (used in the treatment of acute lymphocytic leukemia, choriocarcinoma, mycosis fungiodes breast cancer, head and neck cancer and osteogenic sarcoma) and pteropterin; and the purine analogs such as mercaptopurine and thioguanine which find use in the treatment of acute granulocytic, acute lymphocytic and chronic granulocytic leukemias.

In another embodiment, a compound or salt of this invention is provided in combination with natural product based chemotherapeutic agents such as, without limitation, the vinca alkaloids, e.g., vinblastin (used in the treatment of breast and testicular cancer), vincristine and vindesine; the epipodophylotoxins, e.g., etoposide and teniposide, both of which are useful in the treatment of testicular cancer and Kaposi's sarcoma; the antibiotic chemotherapeutic agents, e.g., daunorubicin, doxorubicin, epirubicin, mitomycin (used to treat stomach, cervix, colon, breast, bladder and pancreatic cancer), dactinomycin, temozolomide, plicamycin, bleomycin (used in the treatment of skin, esophagus and genitourinary tract cancer); and the enzymatic chemotherapeutic agents such as L-asparaginase.

In addition to the above, a compound or salt of this invention may in an exemplary embodiment be used in combination with the platinum coordination complexes (cisplatin, etc.); substituted ureas such as hydroxyurea; methylhydrazine derivatives, e.g., procarbazine; adrenocortical suppressants, e.g., mitotane, aminoglutethimide; and hormone and hormone antagonists such as the adrenocorticosteriods (e.g., prednisone), progestins (e.g., hydroxyprogesterone caproate); estrogens (e.g., diethylstilbesterol); antiestrogens such as tamoxifen; androgens, e.g., testosterone propionate; and aromatase inhibitors (such as anastrozole).

In another embodiment, a combination of a compound of this invention is provided in combination with Camptosar™, Gleevec™, Herceptin™, Endostatin™, Cox-2 inhibitors, Mitoxantrone™ or Paclitaxel™ for the treatment of solid tumor cancers or leukemias such as, without limitation, acute myelogenous (non-lymphocytic) leukemia.

Dosage

Pharmaceutical compositions suitable for use in the present invention include compositions wherein the active ingredients are contained in an amount sufficient to achieve the intended purpose, i.e., the modulation of EGFR activity or the treatment, amelioration or prevention of an EGFR-related disorder, such as cancer.

More specifically, a therapeutically effective amount means an amount of compound effective to prevent, alleviate or ameliorate symptoms of disease or prolong the survival of the subject being treated. For any compound used in the methods of the invention, the therapeutically effective amount or dose can be estimated initially from cell culture assays. Then, the dosage can be formulated for use in animal models so as to achieve a circulating concentration range that includes the IC₅₀ as determined in cell culture (i.e., the concentration of the test compound which achieves a half-maximal inhibition of EGFR activity). Such information can then be used to more accurately determine useful doses in humans.

Toxicity and therapeutic efficacy of the compounds described herein can be determined by standard pharmaceutical procedures in cell cultures or experimental animals, e.g., by determining the IC₅₀ and the LD₅₀ for a subject compound. The data obtained from these cell culture assays and animal studies can be used in formulating a range of dosage for use in humans. The dosage may vary depending upon the dosage form employed and the route of administration utilized. The exact formulation, route of administration and dosage can be chosen by the individual physician in view of the patient's condition. (See, e.g., Fingl, et al., (1975), The Pharmacological Basis of Therapeutics, Ch. 1 p. 1).

Dosage amount and interval may be adjusted individually to provide plasma levels of the active species which are sufficient to maintain the kinase modulating effects. These plasma levels are referred to as minimal effective concentrations (MECs). The MEC will vary for each compound but can be estimated from in vitro data, e.g., the concentration necessary to achieve 50 to 90% inhibition of a kinase may be ascertained using the assays described herein. Preferably, the dosages necessary to achieve the MEC will depend on individual characteristics and route of administration. HPLC assays or bioassays can be used to determine plasma concentrations.

Dosage intervals can also be determined using MEC values. Compounds can in an exemplary embodiment be administered using a regimen that maintains plasma levels above the MEC for 10 to 90% of the time, preferably between 30 to 90% and most preferably between 50 to 90%. In cases of local administration or selective uptake, the effective local concentration of the drug may not be related to plasma concentration and other procedures known in the art may be employed to determine the correct dosage amount and interval.

The amount of a composition administered will, of course, depend on the subject being treated, the severity of the affliction, the manner of administration, the judgment of the prescribing physician, etc.

### Mechanisms of Action

Inhibition of EGFR can occur through a variety of mechanisms. For example, many of the traditionally used anti-EGFR agents exert their effects on EGFR either by binding to the ATP site of the EGFR kinase domain or by down-regulating expression of EGFR to reduce the level of proteins present in cell membranes (Cunningham et al., (2006) Cancer Research, Vol. 15: 7708-15).

The present invention provides novel methods and compositions for inhibition of EGFR, wherein that inhibition occurs by an allosteric mechanism. In contrast to the compositions and methods of the current invention, most currently used therapeutics, such as Erlotinib and Lapatinib, bind directly to the active (ATP-binding) site of the EGFR protein or interfere with the extracellular ligand binding domain. (Lenz, (2006) Oncology. Williston Park, N.Y., Vol. 20, (5 Suppl. 2): 5-13). The present invention relates to compositions and methods in which EGFR activation is modulated through an allosteric mechanism, preferably by preventing the formation of an asymmetric dimer interface between the monomers forming the EGFR dimer.

In one embodiment, the invention provides one or more isolated polypeptides which bind to a kinase domain of an EGFR molecule. In a preferred embodiment, the isolated polypeptides inhibit EGFR activation by preventing the formation of an asymmetric dimer interface between EGFR molecules.

The cytoplasmic EGFR kinase domain corresponds to amino acid residues 672-998 of the human EGFR polypeptide. Studies of EGFR mutants in which the kinase domain has been altered indicates that the kinase domain is an important factor in the survival of cancer cells. (Haber, (2005) Cold Spring Harbor Symposia Quantitative Biology, Vol. 70: 419-26).

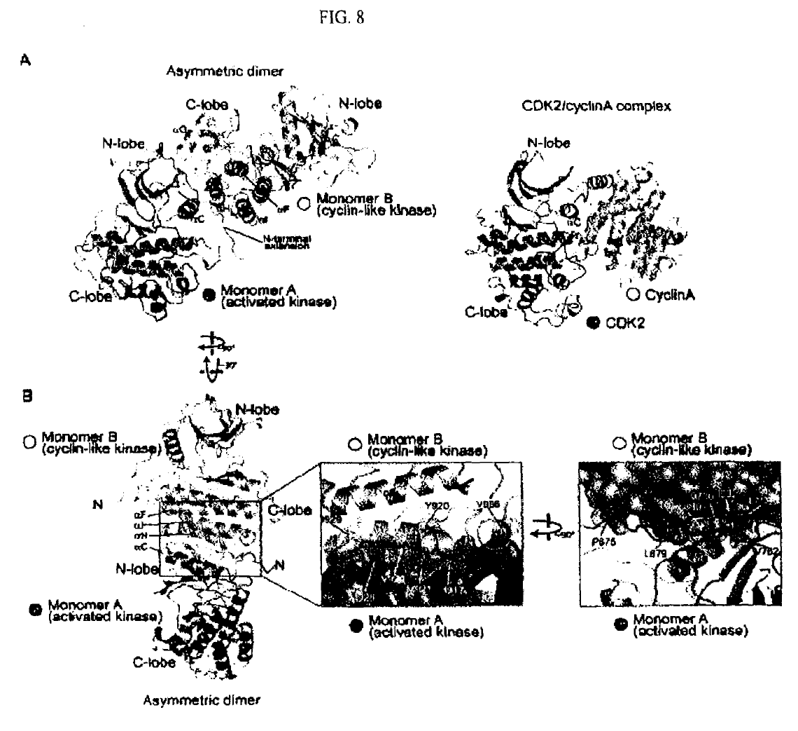

The asymmetric dimer interface is formed by the N-terminal extension (residues 672-685), the C helix, and the loop between strands β4 and β5 of monomer A (the activated kinase domain) and the loop between helices αG and αH, helix αH, and the end of helix αI from monomer B, burying ˜2019 Å² of surface area between them (FIG. 8).

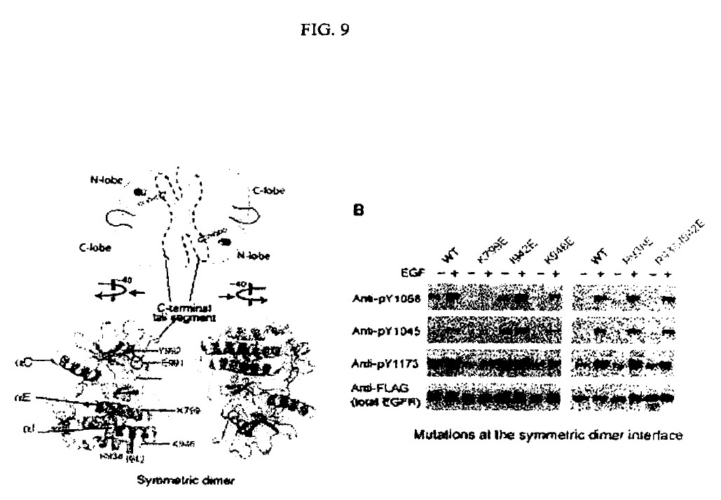

The symmetric dimer interface seen in most crystal structures of the EGFR kinase domain does not play a significant role in the activation of EGFR. A cell transfection assay in which the levels of phosphorylation at three sites in the C-terminal tail of the full-length receptor (Tyr1045, Tyr1068, and Tyr1173) were monitored showed that mutations at the symmetric dimer interface have no effect on the ability of the dimer to activate. (FIG. 9). As described herein, a cell transfection assay includes the monitoring of phosphorylation at specific tyrosine residues using anti-EGFR antibodies. (see, Example V).

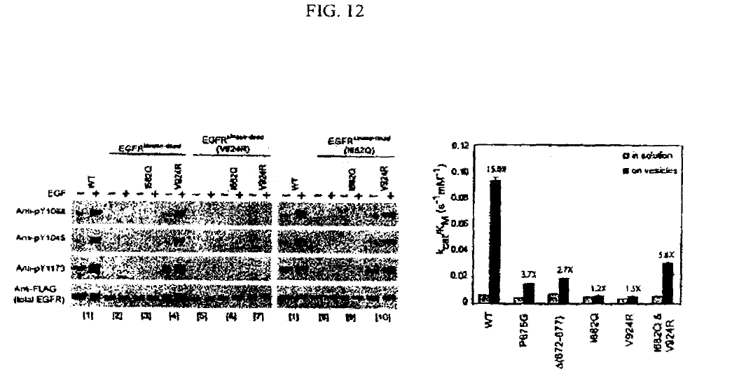

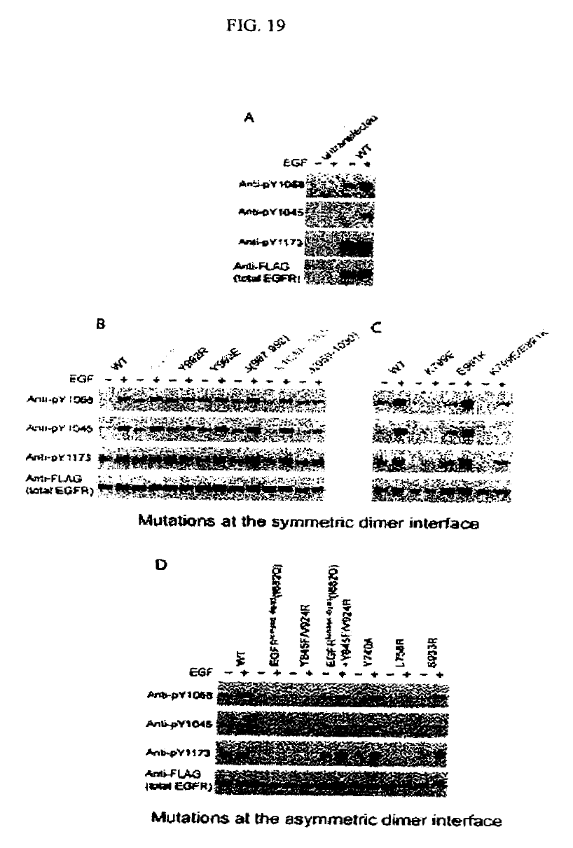

In contrast to the symmetric dimer interface, the asymmetric EGFR dimer interface is vital to the activation of EGFR. Mutation of residues at the asymmetric dimer interface affects auto-phosphorylation of full-length EGFR. Such mutations include P675G, L680A, I682Q, and L736R, which involve residues which are contributed to the interface by monomer A (the activated kinase—see FIG. 8). Additional mutations include I917R, M921R, V924R, and M928R, which involve residues that are contributed to the interface by monomer B (the cyclin-like partner). These mutations diminished the ability of EGFR to phosphorylate three tested auto-phosphorylation sites, either before or after EGF stimulation (FIG. 12 and FIG. 19). A double mutant containing both a C-lobe face mutation and a mutation that replaces the activation loop tyrosine with phenylalanine (Y845F/V924R) showed no significant auto-phosphorylation in a cell transfection assay, but autophosphorylation was rescued by cotransfection with the EGFR^{kinase-dead}(I692Q) mutant. (FIG. 19). These data demonstrate that activation of the receptor is dependent on formation of the asymmetric dimer interface rather than on phosphorylation of the tyrosine residue in the activation loop. The present invention relates to the modulation and interference with this asymmetric dimer interface.

Allosteric Model

An allosteric model predicts that since the dimer interface is asymmetric, an EGFR molecule with a mutation in the C-lobe face of the dimer interface can be activated by another EGFR molecule that has an intact C-lobe interface. Conversely, an EGFR molecule with a mutation in the N-lobe face of the dimer interface (i.e., one that is predicted to be resistant to activation) can act as an activator for another EGFR molecule in which the N-lobe face is intact.

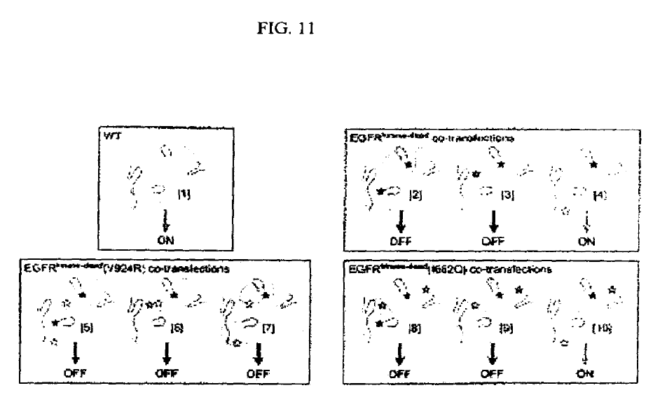

One way to test such a theory is to construct a catalytically dead variant of EGFR in which Asp813 is replaced by asparagine. Asp813 is part of the catalytic base in the kinase domain. Transfection of cells with the “dead” kinase shows that it does not undergo auto-phosphorylation either before or after EGF stimulation (FIG. 11).

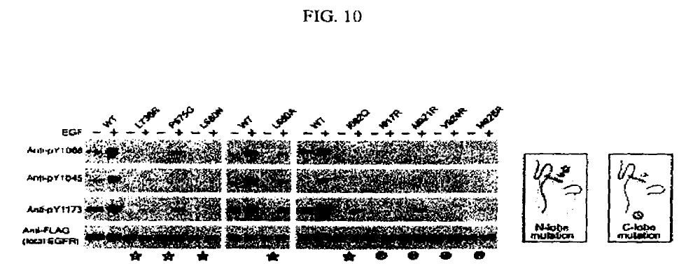

Co-transfection of the dead EGFR with EGFR(I682Q), an N-lobe mutant, does not result in detectable levels of auto-phosphorylation (FIG. 10). In contrast, co-transfection of the dead EGFR with EGFR(V924R) results in robust levels of auto-phosphorylation (FIG. 19). In this case, the EGFR(V924R), a catalytically active C-lobe mutant, has an intact N-lobe face. Although this mutant cannot stimulate itself because of the disrupted C-lobe face, it can be stimulated by the intact C-lobe of the dead EGFR (FIG. 19).

It can be shown that a double mutant, EGFR(Asp813Asx)(I682Q) rescues the auto-phosphorylation of EGFR(V924R) because it has an intact C-lobe that can interact with the intact N-lobe of EGFR(V924R) (FIG. 19). Also consistent with an allosteric model is the inability of EGFR(Asp813Asx)(I682Q) to rescue auto-phosphorylation of EGFR(I682Q) (FIG. 10). In this case, both transfected EGFR molecules have defective N-lobe faces (FIG. 10). Likewise, a double mutant EGFR(Asp813Asx)(V924R), which has a defective C-lobe face, fails to rescue the auto-phosphorylation of either EGFR(I682Q) or EGFR(V924R). (FIG. 10). These results support an allosteric model of activation for the EGFR protein in which the asymmetric dimer interface must form for activation to occur.

Thus, in a preferred aspect, the invention provides inhibitors of EGFR which act at a site other than the active site to allosterically prevent activation of the protein. In a preferred embodiment, this inhibition occurs by preventing the formation of an asymmetric dimer interface between EGFR monomers. Preventing the formation of the asymmetric dimer interface is able to inhibit EGFR, because the interface is vital to the allosteric mechanism of EGFR activation.

### Vesicle Assay System

In one aspect, the invention provides methods for screening for inhibitors of EGFR activation. In a preferred embodiment, these screening methods are able to identify allosteric inhibitors of EGFR.

In a preferred aspect of the invention, a vesicle assay system is used to screen for inhibitors of EGFR activation.

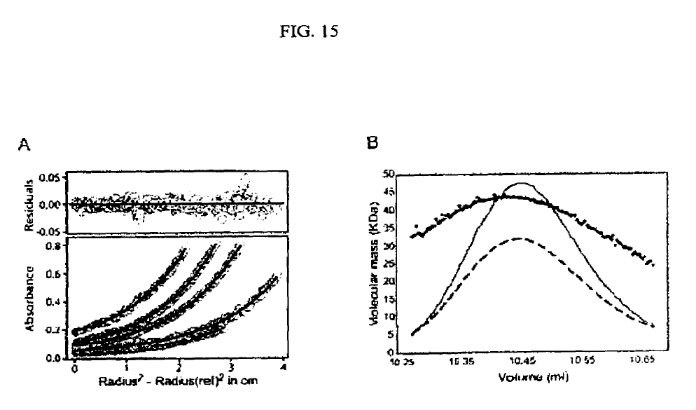

The EGFR kinase domain is monomeric in solution at concentrations up to 50 μM (FIG. 15). The local concentration of kinase domains in a dimeric receptor is estimated to be in the millimolar range. In order to increase the local concentration of the kinase domain in a controlled fashion, one aspect of the invention provides a hexa-histidine tag for the kinase domain to localize it to the surface of vesicles, such as small unilamellar vesicles containing lipids with a nickel-nitrilotriacetate head group (1,2-Dioleoyl-sn-Glycero-3{[N(5-Amino-1-Carboxypentyl)iminodiAcetic Acid]Succinyl} Nickel salt, DOGS-NTA-Ni). The density of the kinase domain on individual vesicles can be controlled, for example, by varying the mole ration of the DOGS-NTA-Ni lipids and the 1,2-Dioleoyl-sn-Glycero-3-Phosphocholine (DOPC) lipids that constituted the vesicles.

The density of DOGS-NTA-Ni lipids in the vesicles is in one embodiment varied from 0.5 to 5.0 mole percent. The dissociation constant for attachment of the His-tagged kinase domain to the vesicle is estimated to be ˜2 μM and the total concentration of DOGS-NTA-Ni lipids is in a preferred embodiment maintained at 12.5 μM to ensure localization of His-tagged protein to the vesicles. The effective local concentration of kinase domains in such a system is in a preferred embodiment approximately in the range of ˜0.4 μM (for 100 nm vesicles containing 0.5 mole % DOGS-NTA-Ni) to ˜4 μM (for 5 mole % DOGS-NTA-Ni).

In one aspect of the invention, a method utilizing a vesicle assay system is provided for screening for potential inhibitors of EGFR activation. In this method, an isolated polypeptide corresponding to an EGFR kinase domain is attached to the surface of a vesicle, which is in an exemplary embodiment a lipid vesicle. This attachment forms a conjugated polypeptide. In an exemplary embodiment, the activity of the conjugated polypeptide is determined using techniques known in the art, such as Western blot analysis. The conjugated polypeptide is then contacted with a test compound, and the activity of the conjugated polypeptide is determined after contact with the test compound. If a comparison of the activity of the conjugated polypeptide before and after contact with the test compound shows a difference, namely that the activity decreases upon contact with the test compound, then the test compound is identified as an inhibitor of EGFR activation.

In one embodiment, the invention provides a test compound which comprises a polypeptide of about 75 or fewer amino acid residues in length. In a further embodiment, the invention provides a test compound which is at least about 85% homologous to an amino acid sequence selected from SEQ ID NOs: 1-9. In a still further embodiment, the invention provides a test compound which is at least about 90% homologous to SEQ ID NOs: 1-9. In a still further embodiment, the invention provides a test compound which is at least about 95% homologous to SEQ ID NO: 1-9s. In a still further embodiment, the invention provides a test compound which is at least about 98% homologous to SEQ ID NOs: 1-9. In a still further embodiment, the invention provides a test compound which is at least about 99% homologous to SEQ ID NOs: 1-9. In a still further embodiment, the invention provides a test compound which is at least about 100% homologous to SEQ ID NOs: 1-9.

An assay that measures the functional property of a molecule, such as the catalytic activity of a protein, is a functional assay. In one aspect, the invention provides a functional assay in which mutant EGFR kinase domain molecules are expressed in host cells and then purified from those host cells. These mutant EGFR kinase domain molecules are then localized to surfaces of vesicles, which are, in an exemplary embodiment, lipid vesicles. The catalytic activity of the EGFR kinase molecules can then measured in such a vesicle assay system. The catalytic activity of the mutant EGFR kinase domain molecules is compared to the catalytic activity of wildtype EGFR kinase domain molecules in the same vesicle system in order to determine the functional effects of the mutations present in the mutant EGFR kinase domain molecules.

In one embodiment, the invention provides a method for localizing the mutant EGFR kinase domain molecules to the surfaces of lipid vesicles which utilizes a tag molecule, and in a further embodiment, this tag molecule does not interfere with the catalytic activity of the attached mutant or wildtype EGFR kinase domain molecule. In a further embodiment of the invention, the tag molecule is a hexa-histidine tag.

### Binding Assays

Binding assays can be used to determine whether there is an interaction between part of a molecule and a test compound, a ligand, another similar molecule, etc. In one aspect, the invention provides a method of screening for compounds which bind to the kinase domain of EGFR. This method involves determining the ability of a potential binding agent to compete with a polypeptide which has an amino acid sequence selected from SEQ ID NOs: 1-9.

In one embodiment, the polypeptide is radioactively or fluorescently labeled and mixed with EGFR kinase domain to form a protein/polypeptide complex. Any compounds can be added into the solution containing the complex, and the release of the labeled polypeptide from the complex can be monitored. Compounds causing the release are then identified as potential inhibitors that are able to bind to the same are on the kinase as the labeled polypeptide. These compounds can then in a further embodiment be assessed using the vesicle assay system of the present invention to distinguish traditional ATP-competitive inhibitors from novel inhibitors with allosteric mechanisms of action. Novel inhibitors will only inhibit the activation of the kinase activity in the vesicle assay, whereas traditional ATP-competitive inhibitors inhibit basal activity in solution as well as in the vesicle assay system.

Those skilled in the art will recognize a wide variety of fluorescent reporter molecules that can be used in the present invention, including, but not limited to, fluorescently labeled biomolecules such as proteins, phospholipids and DNA hybridizing probes. Similarly, fluorescent reagents specifically synthesized with particular chemical properties of binding or association can be used as fluorescent reporter molecules (Barak et al., (1997) Journal of Biological Chemistry, Vol. 272: 27497-27500; Southwick et al., (1990) Cytometry, Vol. 11: 418-30; Tsien, (1989) Methods in Cell Biology, Vol. 29 Taylor and Wang (eds.): 127-156). Fluorescently labeled antibodies are particularly useful reporter molecules due to their high degree of specificity for attaching to a single molecular target in a mixture of molecules as complex as a cell or tissue.

Luminescent probes can be synthesized within the living cell or can be transported into the cell via several non-mechanical modes including diffusion, facilitated or active transport, signal-sequence-mediated transport, and endocytotic or pinocytotic uptake. Mechanical bulk loading methods, which are well known in the art, can also be used to load luminescent probes into living cells (Barber et al., (1996) Neuroscience Letter, Vol. 207, pages 17-20; Bright et al., (1996) Cytometry, Vol. 24: 226-33). These methods include electroporation and other mechanical methods such as scrape-loading, bead-loading, impact-loading, syringe-loading, hypertonic and hypotonic loading. Additionally, cells can be genetically engineered to express reporter molecules, such as GFP, coupled to a protein of interest (Chalfie et al., U.S. Pat. No. 5,491,084; Cubitt et al., (1995) Trends in Biochemical Science, Vol. 20: 448-55).

Those skilled in the art will recognize a wide variety of ways to measure fluorescence. For example, some fluorescent reporter molecules exhibit a change in excitation or emission spectra, some exhibit resonance energy transfer where one fluorescent reporter loses fluorescence, while a second gains in fluorescence, some exhibit a loss (quenching) or appearance of fluorescence, while some report rotational movements (Giuliano et al., (1995) Annual Review of Biophysics and Biomolecular Structure, Vol. 24: 405-3434; Giuliano et al., (1995) Methods in Neuroscience, Vol. 27: 1-16).

### Targeted Drug Discovery

In order to identify compounds which can serve as potential therapeutics for EGFR-activation related diseases, methods of targeted drug discovery utilizing structural information of the protein are provided the present invention. Although the following discussion relies in part on a description of embodiments utilizing HER1, it will be appreciated that any member of the EGFR family is encompassed by the embodiments described herein.

In one aspect, the invention provides a method in which cells expressing EGFR are contacted with a compound of this invention (or its salt), and these cells are then monitored for any effect that the compound has on them. The effect may be any observable, either to the naked eye or through the use of instrumentation, change or absence of change in a cell phenotype. The change or absence of change in the cell phenotype monitored may be, for example, without limitation, a change or absence of change in the catalytic activity of EGFR in the cells or a change or absence of change in the interaction of the protein with a natural binding partner.

In one aspect, the invention provides a method for identifying compounds which modulate activation of EGFR. In a preferred aspect of the invention, the ability of a compound to modulate activation of EGFR is predicted based on a theoretically predicted interaction between the compound and an X-ray crystal structure of an EGFR kinase domain, or an X-ray crystal structure of an EGFR kinase domain co-crystallized with a control compound. In one embodiment of the invention, the control compound co-crystallized with the EGFR kinase domain has an amino acid sequence selected from SEQ ID NOs: 1-9. In a further embodiment, the invention provides a method whereby a plurality of atomic coordinates is obtained from structural analysis of the co-crystallized molecules.

In another aspect, the invention provides a method of targeted drug discovery in which the structural information is obtained of an EGFR kinase domain co-crystallized with a control molecule, and residues of the EGFR kinase domain which interact with the control molecule are identified. The structural information from the crystal structure along with the residues of interaction between the kinase domain and the control molecule are compared to a database of potential therapeutics. Potential therapeutics are selected from the database using the structural information to narrow the search parameters and identify the therapeutics most likely to interact with the EGFR kinase domain in the same manner as the control molecule.

In one embodiment, the control molecule used in the above method of targeted drug discovery is an isolated polypeptide. In a further embodiment, the isolated polypeptide comprises an amino acid sequence selected from SEQ ID NOs: 1-9.

In another aspect of the invention, a method is provided for identifying effective therapeutics using a vesicle assay system, in which a decrease in EGFR dimer formation identifies an effective therapeutic. In one embodiment of the invention, the inhibition of dimer formation occurs by binding of the therapeutic to a site on the C-terminal lobe of a kinase domain of an EGFR polypeptide, wherein the site is distal to the ATP binding site.

Assay Based on Interference with Kinase Domain Dimerization

In a preferred aspect, the present invention provides methods of screening for inhibitors of EGFR. As discussed herein, the dimer interface of EGFR includes the C-terminal lobe of one kinase domain which interacts with the N-terminal lobe of the other, and stabilizes the active state in the latter. Dimer formation is necessary for signaling activity of EGFR even when the kinase domain is rendered constitutively active in terms of its ability to catalyze phosphate transfer reactions. This is demonstrated by the fact that a mutant form of the EGF receptor (EGFR L834R) does not show signaling activity in the absence of EGF, despite the fact that its isolated kinase domain is fully active as a monomer in the in vitro assays. (see, e.g., Zhang et al., (2007) Nature). Therefore, in accordance with the invention, methods are provided for identifying molecules capable of inhibiting asymmetric dimer formation, thus limiting activity of the wild type and kinase-activated EGF receptors.

In one embodiment, assays of the invention screen for small molecule inhibitors that disrupt asymmetric dimerization of EGFR by binding to the N-lobe of the kinase and thereby preventing its interaction with the C-lobe of the second monomer in a dimer. Such inhibitors of protein-protein interactions are normally very difficult to identify. By recognizing that the EGFR kinase domain has very low basal activity as a monomer, the present invention provides an assay that searches for small molecules that activate the isolated kinase domain of EGFR. The transition between the inactive and active forms of the EGFR kinase domain involves a rotation of an alpha helix in the kinase domain (named helix C). Upon activation, a hydrophobic pocket opens up between helix C and the main body of the kinase domain. Normally this hydrophobic pocket is filled by residues presented by the “activator” kinase domain in the asymmetric dimer. In a preferred embodiment, the assay is used to screen for small molecules that fill this hydrophobic pocket and therefore switch on the kinase activity of the normally inactive isolated EGFR kinase domain.

Basing the strategy on a search for kinase activators provides a tremendous advantage in the inhibitor screen, because it avoids the false positive results that are confounding problem of normal inhibitor assays. It also avoids the discovery of compounds that inhibit the kinase domain by binding to the ATP binding site—this site is a common binding site for small molecules, but molecules that bind here would not block the asymmetric dimer formation.

In the screen the activity of the wild type kinase domain towards a substrate, peptide in solution will be used as readout. The feasibility of the screen is based on the fact that in solution, the activity of the wild type kinase domain is low due to its monomeric state and the inability to stabilize the active conformation in the asymmetric dimer. This activity is 15 fold higher for the mutant EGFR kinase domain (EGFR L834R), which is in the active conformation in the absence of dimerization. The increase in EGFR kinase domain activity as a result of the compound binding should be therefore easily detected.

The compounds identified in the screen should act as inhibitors of full length EGFR activity upon introduction to the cells due to their ability to prevent EGFR kinase domain dimerization. This prediction is based on the aforementioned observation that the kinase domain activating EGFR mutant (L834R) is inactive when its dimerization is prevented in the full length receptor. In another scenario, the identified compounds can be further modified by structure-based design to directly inhibit kinase domain activity while retaining their ability to interfere with dimerization of the kinase domains.

The allosteric activation of the kinase with a small molecule compound has been found in the case of phosphoinositide-dependent protein kinase 1 (PDK1), providing proof of the principle that such compounds can bind to protein surfaces and induce large conformational changes. (see Engel et al., (2006) Embo J., 25:5469-80). In addition to PDK1 binding compound, a growing number of small molecule inhibitors of protein-protein interactions is being successfully identified and validated as functional inhibitors in vivo. (see Arkin et al., (2004), Nat Rev Drug Discov., 3: 301-17).

Assays according to the invention can thus target small molecule inhibitors of EGFR dimerization, providing a novel approach to target EGFR signaling in disease. Inhibitors found using assays of the invention could significantly enhance the unsatisfying performance of the current anti-EGFR therapeutics that include tyrosine kinase inhibitors. In addition, due to conservation of the dimerization interface between different members of erbB family, such compounds may also serve as potent inhibitors of the signaling crosstalk between HER1, HER2 and HER3. Such crosstalk has been implicated in promotion of cancer growth and drug resistance. (Sergina et al., (2007) Nature).

### Inhibition of EGF Receptor by Binding MIG6 to an Activating Kinase Domain Interface

Signaling by EGFR molecules that contain constitutively active kinase domains requires formation of the asymmetric dimer, underscoring the importance of dimer interface blockage in MIG6-mediated inhibition.

Before activation, the EGFR kinase domain is in an autoinhibited conformation that resembles that of inactive cyclin-dependent kinases (CDKs) and the Src family kinases2,6. Conversion to the active form requires interactions between the distal surface of the C lobe ofone kinase domain and the amino-terminal lobe (N lobe) of the other in the asymmetric activating dimer. This conformational change resembles closely the activation switch induced in CDKs by cyclins7, even though the Clobe of the EGFR kinase domain is structurally unrelated to cyclins.

If the cyclin/CDK-like asymmetric dimer is indeed critical for EGFR activation, then the modulation of this interaction might underlie naturally occurring mechanisms of EGFR regulation. We looked for protein inhibitors of EGFR that are known to function by interacting with the intracellular portions of the receptor. One such protein is MIG6 (or receptor-associated late transducer, RALT, the gene for which is also named gene 33), which is a feedback inhibitor of both EGFR and ERBB2. (see Hackel et al., (2001) Biol. Chem. 382:1649-162; Fiorentino et al., (2000), Mol. Cell. Bio., 20:7735-7750). MIG6 inhibits EGFR-mediated signals in mouse skin, and deletion of the MIG6 gene leads to hyper-activation of EGFR.

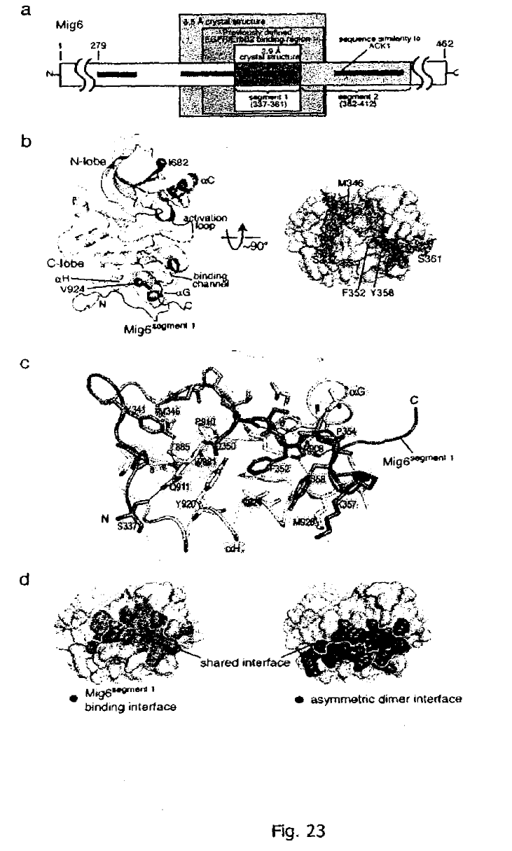

The N-terminal region of MIG6 is not implicated in EGFR inhibition (FIG. 23a). The C-terminal region shows sequence similarity to only a non-catalytic region of the ACK1 tyrosine kinase (FIG. 23a), which also binds to the EGFR cytoplasmic domain. A segment within this region of MIG6 (residues 323-372) is critical for EGFR and ERBB2 binding (FIG. 23a). We determined the crystal structure of a 60-residue fragment spanning this segment (residues 315-374) bound to the EGFR kinase domain. This structure and structures of EGFR complexed to two overlapping 40- and 25-residue fragments (residues 325-364 and 340-364) define a 25-residue epitope of MIG6 that binds to the EGFR kinase domain (residues 337-361, denoted MIG6(segment 1). The structure of the 40-residue peptide complex has been determined at 2.9 Å resolution.

The EGFR kinase domain bound to MIG6(segment 1) adopts the Src/CDK-like inactive conformation, and not the active conformation normally seen in crystals of the kinase domain (FIG. 23b). The interface, which buries 1,800 Å² of surface area, involves an extended conformation of the MIG6 peptide and disparate binding elements on the kinase domain (FIG. 23 b and c). MIG6(segment 1) lies within a shallow depression on the distal surface of the C lobe of the kinase domain, formed by helices αG and αH and the loops connecting helices αF-αG, αG-αH and αH-αI. The interactions are mainly polar, although a few hydrophobic residues from helix αH contribute to the interface.

The footprint of MIG6(segment 1) on the kinase domain overlaps the cyclin-like face of the kinase domain in the asymmetric kinase domain dimer, and so binding of MIG6 to an EGFR kinase domain will prevent it from acting as a cyclin-like activator for other kinase domains (FIG. 23). Residues in EGFR located at the MIG6(segment 1)-binding interface are conserved, suggesting that MIG6 will also bind to other EGFR family members.

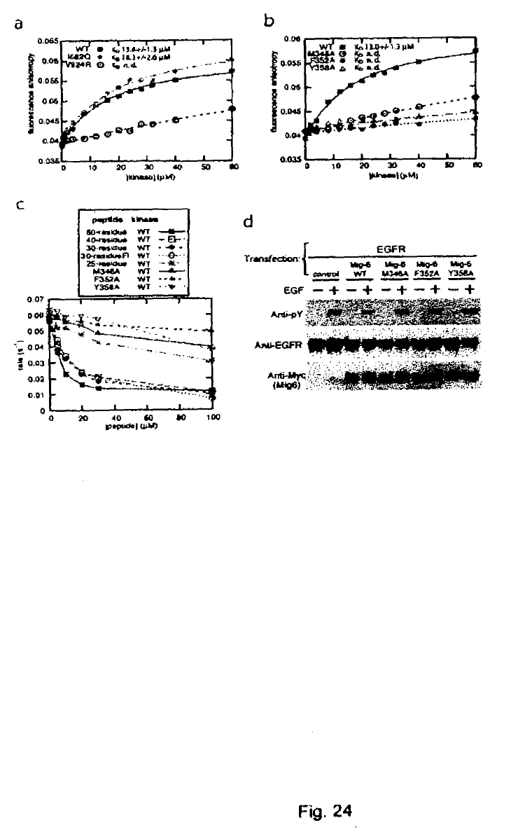

MIG6(segment 1) binds to the EGFR kinase domain with micromolar affinity. The dissociation constant for a 30-residue fluorescein-labelled MIG6 peptide (residues 334-363, spanning the entire binding epitope of segment 1) is 13.061.3 μM (FIG. 24a). Val 924 in the C lobe of the kinase domain is located in the centre of the asymmetric kinase domain dimer interface and also participates in the interaction between the kinase domain and MIG6(segment 1)2 (FIGS. 23b, c). A V924R mutation in the kinase domain abolishes peptide binding (FIG. 24a). Met 346, Phe 352 and Tyr 358 in MIG6 are within the kinase/MIG6(segment 1) interface (FIG. 23c), and mutation of any of these residues also abrogates binding (FIG. 24b).

The EGFR kinase domain has very low activity in solution, but is activated on increasing its local concentration by tethering it to lipid vesicles, which promotes the formation of the asymmetric dimer. Various MIG6 peptides that contain segment 1 inhibit the activity of the kinase domain attached to lipid vesicles, with half maximal inhibitory concentration (IC50) values of ˜10 μM (FIG. 24c). A 25-residue peptide (residues 340-364) that lacks 3 residues in the N-terminal portion of MIG6(segment 1), is much less potent (FIG. 24c). Peptides that contain mutations that disrupt the binding interface (M346A, F352A and Y358A) do not inhibit kinase activity significantly (FIG. 24c). An EGFR kinase domain bearing an I682Q mutation is not stimulated by concentration at the membrane because it is unable to form the asymmetric dimer. The basal activity of this mutant in solution is not inhibited by MIG6(segment 1), which has the same binding affinity for this mutation as for the wild-type kinase domain (FIG. 24a). Thus, MIG6(segment 1) is only able to inhibit the kinase domain in the context of asymmetric dimer formation.

We tested the inhibition of EGFR autophosphorylation by full-length MIG6 in a cell-based assay. Co-expression of the wild-type MIG6 with EGFR decreases the EGF-induced autophosphorylation of EGFR, whereas introduction of individual mutations in MIG6(segment 1) (M346A, F352A or Y358A) abolishes this effect (FIG. 24d), confirming that segment 1 is important for inhibition of EGFR by full-length MIG6.

An intriguing property of MIG6 is its ability to bind more tightly to activated EGFR than to the unliganded receptor. MIG6(segment 1) alone cannot confer this property, because the kinase residues that interact with it do not change conformation on activation. The C terminus of MIG6(segment 1) is located within a channel leading into the kinase active site (FIG. 23b), used by peptidic inhibitors of protein kinases that interact directly with the active sites. The region of MIG6 that is C-terminal to segment 1 (segment 2, FIG. 23a) contains a region of strong homology to ACK1 (also known as TNK2). Because MIG6 and ACK1 are both sensitive to the activation state of EGFR, there may be specific interactions between segment 2 and the activation loop and/or the N lobe of the kinase domain.

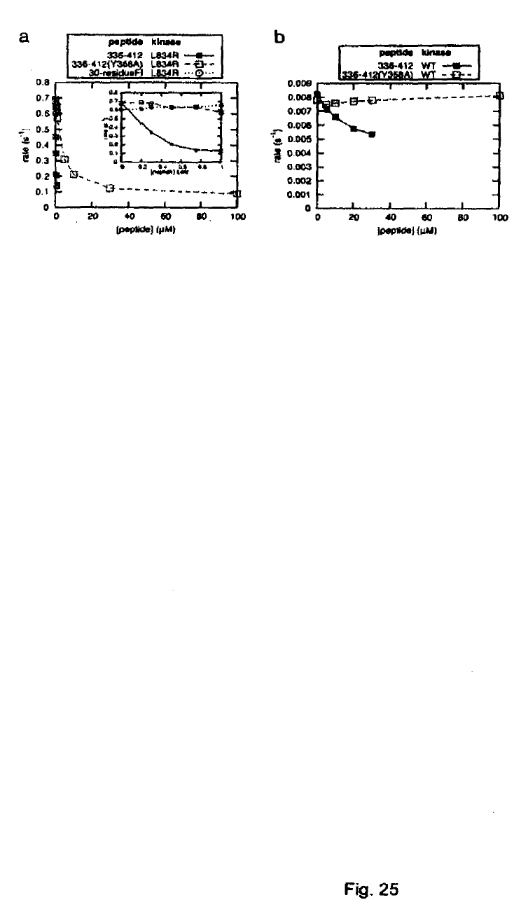

To test the role of segment 2, we produced a longer peptide (residues 336-412, MIG6(segments 1-2)), and analyzed its effect on a variant of the EGFR kinase domain that contains a mutation (L834R) that renders it constitutively active in the absence of concentration on vesicles. MIG6(segments 1-2) inhibits this mutant kinase domain with an IC₅₀ value of ˜200 nM (FIG. 25a). MIG6(segments 1-2) bearing a mutation within segment 1 (Y358A) inhibited L834R much less efficiently (IC₅₀˜5 μM). MIG6(segment 1) (the 30-residue peptide) did not inhibit this mutant kinase, consistent with its dimerization-independent activity. Interestingly, MIG6(segments 1-2) seems to be much less potent in inhibiting the basal activity of the wild-type kinase domain in solution, and MIG6(segments 1-2) bearing a mutation in segment 1 (Y358A) does not show any inhibition under the same conditions (FIG. 25b). These results suggest that segment 2 is responsible for the inhibition of the activated EGFR kinase domain, and that both segments 1 and 2 are important for the high potency of inhibition.

Could MIG6 function by binding primarily to the activated kinase in an asymmetric kinase domain dimer, and not to the cyclin-like activator kinase? The MIG6(segment 1) interaction would then be important for anchorage of MIG6 to EGFR, but not directly relevant for shutting down kinase activity. Such a role may be operative in auto-inhibition of ACK1, the kinase domain of which has a conserved segment-1-binding surface, with the MIG6 homologous segments present within the same protein. It is also possible that the asymmetric EGFR dimer will dissociate, and that activated kinase molecules can subsequently serve as cyclin-like activators. This may facilitate the lateral propagation of EGFR activation, which can spread across the cell surface even when EGF is localized to a small region. The interaction between MIG6(segment 1) and the kinase domain would block further transmission of the activating signal.

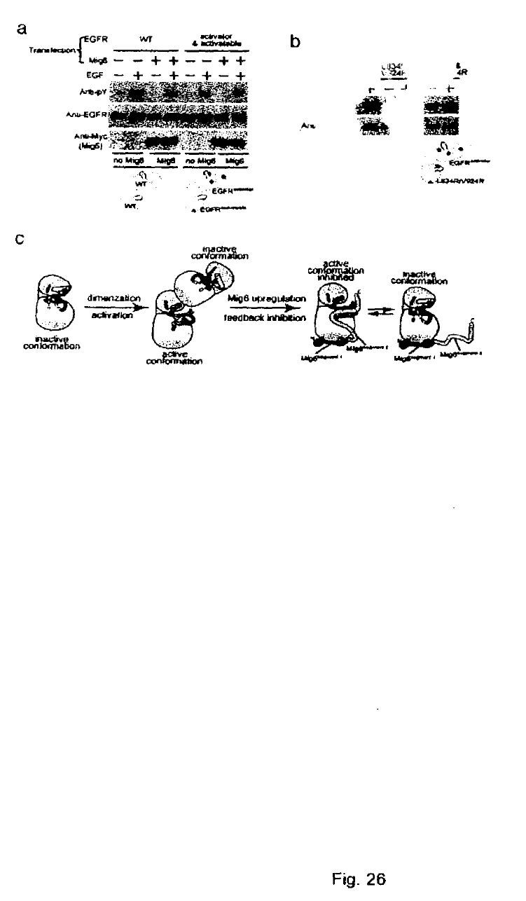

To examine this potential, we co-transfected cells with two variants of EGFR. One form (EGFR(activator)) resembles ERBB3 in that it is catalytically inactive (the catalytic base, Asp 813, is mutated to Asn) but can serve as a cyclin-like activator. To promote interaction with MIG6, we introduced the L834R mutation, which destabilizes the inactive conformation, into the EGFR(activator). To prevent EGFR(activator) from assuming the ‘activated’ position in the asymmetric dimer, we also introduced the I682Q mutation. The second EGFR variant (EGFR(activatable)) is catalytically active, but has the V924R mutation, which prevents it from serving as an activator. We tested the effects of MIG6 on EGFR phosphorylation in cotransfections with these two variants. The results show that EGFR(activator) can activate EGFR(activatable) in the presence of EGF (FIG. 26a), consistent with previous findings. (see Zhang et al., (2006) Cell, 125: 1137-49, which is hereby expressly incorporated by reference in its entirety). Cotransfection of MIG6 with EGFR(activator) and EGFR(activatable) suppresses this activation (FIG. 26a). MIG6(segment 1) does not bind to the kinase domain bearing the V924R mutation, and an intact MIG6(segment 1) is required for inhibition of EGFR in cellular assays (FIG. 24). We therefore interpret the results of the triple transfection experiment (FIG. 26a) to mean that MIG6 binds to EGFR(activator) and prevents the activation of EGFR(activatable).

Full-length EGFR bearing the activating L834R mutation is not fully phosphorylated in cells, suggesting that the formation of the asymmetric dimer is still required for robust autophosphorylation even when the kinase domain is rendered constitutively active. We confirmed this by introducing the V924R mutation, which prevents the kinase domain from serving as the cyclin-like activator, into EGFR with a constitutively active kinase domain (L834R/V924R). EGFR(L834R/V924R) fails to undergo autophosphorylation (FIG. 26b), although the kinase activity of this double mutant is comparable to that of the kinase domain bearing the single activating mutation (L834R). EGF-stimulated autophosphorylation is restored when this double mutant is co-transfected with the kinase-dead EGFR(activator) (FIG. 26b). These results further underscore the importance of blockage of the asymmetric dimer interface by MIG6, because it can prevent both the activation of kinase domains and downstream signaling by activated kinase domains.

Without being limited to this theory, it is possible that in one aspect of the invention, MIG6 uses a double-headed mechanism for inhibiting EGFR, with the blockage of the asymmetric cyclin/CDK-like dimer being a particularly interesting aspect of the inhibition (FIG. 26c). This mechanism provides direct confirmation of the critical role of the asymmetric kinase domain dimer in the activation of EGFR family receptors. In addition, our results suggest an approach for the development of a new class of inhibitors that act by binding to the cyclin-like face of the C-lobe of the kinase domains of this family. This region is not conserved in other protein kinases, and so such inhibitors may enable the development of cancer therapies with a high degree of specificity towards EGFR family members.

The wild-type and mutant forms of the EGFR kinase domain were expressed and purified using methods known in the art and described in Zhang et al., (2006). The 60-residue MIG6 peptide was expressed in bacteria as a glutathione S-transferase (GST)-fusion protein, purified and treated with the TEV protease to remove the GST-moiety. The wild-type and Y358A mutant MIG6(segments 1-2) peptides were fused to a Trp DLE leader peptide and expressed as inclusion bodies and purified as described. All other MIG6 peptides were produced using solid phase synthesis. The EGFR kinase domains (wild-type and the K799E mutant) were co-crystallized with the 60-residue, 25-residue and 40-residue MIG6 peptides and the structures were solved by molecular replacement using a structure of the EGFR kinase domain adopting the Src/CDK-like inactive conformation (PDB entry: 2GS7) as the search model. The binding affinities between the kinase domain and fluorescein-labeled MIG6 peptides were measured by monitoring the change of fluorescence anisotropy during the titration and fitting the data to a single-site binding model. Kinase assays in solution and on vesicle were performed using methods known in the art and described herein. Cell-based inhibition assays were performed using Cos-7 cells co-transfected with constructs containing full-length EGFR and MIG6.

The 60-residue peptide was expressed as a GST-fusion in Escherichia coli BL21 (DE3) by using pGEX6p1 (Amersham) (BamHI/XhoI) and purified using a glutathione Sepharose column. The protein was treated with the PreScission protease to release the MIG6 peptide, which was further purified using a Hitrap SP column (Amersham). The longer peptides (336-412 and 336-412(Y358A)) were cloned as Trp DLE fusions and expressed as inclusion bodies as described previously (Conti et al., (2000), Structure, 8:329-338). To prevent cleavage of the MIG6 peptides by subsequent cyanogen bromide treatment the single methionine in these peptides (M346) was mutated to leucine. This mutation does not affect the binding to the EGFR kinase domain significantly. The fusion proteins were cleaved with cyanogen bromide and the released MIG6 peptides were purified. All other MIG6 peptides were synthesized using solid-phase peptide synthesis using the Fmoc strategy with Wang resin on a Protein Technologies PS3 synthesizer. The peptide identities were confirmed by mass spectrometry.

The wild-type kinase domain was first co-crystallized with the 60-residue MIG6 peptide and the structure was determined at 3.5 Å resolution. This revealed that a ˜25-residue segment of the peptide is bound to the distal surface of the C lobe of the EGFR kinase domain and that the rest of the peptide is disordered. A 25-residue peptide (residues 340-364 in MIG6) was designed on the basis of the initial structure and co-crystallized with both the wild-type and a mutant (K799E) form of the EGFR kinase domain. The K799E mutation does not affect the conformation of the kinase domain or its interaction with MIG6(segment 1), but crystals of this mutant kinase domain in complex with the peptide diffracted X-rays to higher resolution. The structure shows that this 25-residue peptide lacks the N-terminal part of the kinase binding epitope. This peptide was then extended to include residues 325-364 in MIG6 (the 40-residue peptide) and co-crystallized with the EGFR(K799E) kinase domain. The structure of this peptide—kinase domain complex was determined at 2.9 Å°. There are four kinase domains in the asymmetric unit, all of which adopt the same conformation. Two of the four kinase domains are bound to the MIG6 peptide, and the MIG6 binding surfaces of the other two are occupied by crystal contacts.

Fluorescein-labelled 30-residue wild-type, M346L, M346A, F352A and Y358A MIG6 peptides were diluted to final concentrations of 5, 8, 3.1, 3.5 and 2.7 μM in a buffer containing 10 mM Tris, 50 mM NaCl and 2 mM DTT, pH7.5. These peptides in the cuvette were then titrated with the wild-type or mutant forms of the EGFR kinase domain at 20 uC. For the competition assays, the labeled 30-mer wild-type peptide (5 mM) and kinase domain (60 mM) were mixed and titrated with unlabelled competitor peptides. The fluorescence anisotropy at each titration step was monitored. The I682Q and K799E mutant kinases used in the binding assays contain the N-terminal 63H is tag and linker fragment before the kinase domain, whereas this N-terminal fragment in the wild-type and V924R mutant kinases was removed by Tobacco Etch Virus protease treatment.

Kinase assays were performed using methods known in the art and described herein. The substrate peptide was kept at 1 mM in all the experiments. The reported rates are the initial velocities normalized by the kinase concentrations. The wild-type kinase concentrations in the vesicle-based and solution-based assays were 3.5 and 14 mM respectively. Preliminary experiments showed that peptide 336-412 (MIG6(segments 1-2) inhibited the L834R mutant kinase much more strongly and also caused precipitation when both the kinase and the peptide were at high concentrations. We therefore reduced the concentration of L834R in the assays to 200 nM. The higher intrinsic activity of this mutant and usage of MnCl2 at 10 mM instead of MgCl2 allowed us to measure kinase activity at such a low kinase concentration.

For cell-based assays, Cos-7 cells were co-transfected using Fugene 6 (Roche) with the DNA encoding the N-terminal Flag-tagged EGFR in pcDNA3.1 constructs and the wild-type or mutants of the MIG6 genes with a C-terminal Myc tag (also in pcDNA3.1). Cells were cultured for 36 h after transfection and serum-starved for 12 h. Cells were treated with EGF (50 ng ml⁻¹) for ˜5 min at 37° C., lysed and subjected to western blot analyses. The levels of total EGFR, EGFR autophosphorylation and MIG6 were probed using the anti-EGFR antibody SC03 (Santa Cruz), anti-phosphotyrosine antibody 4G10 (Upstate) and an anti-Myc antibody (Cell Signalling), respectively.

### Purification of Expressed Proteins

One aspect of the present invention utilizes proteins and polypeptides corresponding to the EGFR kinase domain or to the Mig-6 protein. These proteins and polypeptides are used in assays, as inhibitors, or as starting material for crystallization in accordance with various aspects of the present invention. These proteins and polypeptides can be expressed in host cells and purified using techniques described herein and known in the art.

In one embodiment, protein and fragments thereof can be isolated and purified from a reaction mixture by means of peptide separation, for example, by extraction, precipitation, electrophoresis and various forms of chromatography. The proteins of this invention can be obtained in varying degrees of purity depending upon the desired use. Purification can be accomplished by use of protein purification techniques or known in the art.

### Crystallization Techniques

Crystal structures described herein are derived using standard techniques known in the art. In a preferred embodiment, crystal structures are generated using X-ray crystallography to generate electron density maps. (see Example IV).

Protein for crystals and assays described herein can be produced using expression and purification techniques described herein and known in the art. For example, high level expression of EGFR or Mig-6 can be obtained in suitable expression hosts such as E. coli. Yeast and other eukaryotic expression systems can also be used.

Crystals may be grown or formed by any suitable method, including drop vapor diffusion, batch, liquid bridge, and dialysis, and under any suitable conditions. Crystallization by drop vapor diffusion is often preferable. In addition, those of skill in the art will appreciate that crystallization conditions may be varied. Various methods of crystallizing polypeptides are generally known in the art. See, for example, WO 95/35367, WO 97/15588, EP 646 599 A2, GB 2 306 961 A, and WO 97/08300.

In one embodiment of the invention, a DNA construct comprising EGFR residues 672-998 is provided. In an exemplary embodiment, the DNA construct comprising EGFR residues 672-998 also includes an N-terminal 6-His tag, a linker and a cleavage site for Tobacco Etch Virus protease. In a further embodiment, the DNA construct is expressed in Sf9, CHO or E. coli cells. The expressed protein is then purified using techniques known in the art.

After purification, the expressed protein can be stored in a crystallization buffer. Suitable crystallization buffers, for example, include: 0.1 M Na Acetate pH 5.3, 0.2 M CaCl₂, 30% v/v Ethanol; 0.1 M Na Citrate pH 5.0, 40% v/v Ethanol; 0.1 M Na Citrate pH 8.7, 20% w/v PEG 4000, 20% v/v Isopropanol; and 0.1 M Na Citrate pH 5.4, 20% w/v PEG 4000, 20% v/v Isopropanol. The sample can be incubated at a temperature ranging from about 4 to 20 degrees Celsius until a crystalline precipitate is formed. Seeds from the crystalline precipitate obtained, as whole crystals or as crushed crystal suspensions, are transferred, along with a suitable crystallization promoter, such as hair of rabbit, to a solution of concentrated substrate in a crystallization buffer in order to allow crystals suitable for X-ray data collection to form.

X-Ray Diffraction

Another aspect of the invention relates to the structure of EGFR, particularly the structure of the EGFR kinase domain. The structure of the kinase domain can be determined utilizing a crystal comprising a polypeptide as described above. According to a preferred embodiment of the present invention, the structure of EGFR, and particularly the EGFR kinase domain, is determined using X-ray crystallography. Any suitable X-ray diffraction method for obtaining three-dimensional structural coordinates of a polypeptide may be used.

Methods of Using X-Ray Diffraction Coordinates

The invention also relates to use of the structural coordinates obtained from the above described X-ray diffraction studies of the EGFR kinase domain. The coordinates may be used, with the aid of computer analysis, to determine the structure of the protein, which can include the secondary and tertiary structure. The EGFR kinase domain structural coordinates can also be used to develop, design, and/or screen compounds that associate with EGFR. As used herein, “associate” means that the compound may bind to or interact with EGFR ionically, covalently, by hydrogen bond, van der Waals interaction, salt bridges, steric interaction, hydrophilic interactions and hydrophobic interaction. The term “associate” also encompasses associations with any portion of the EGFR kinase domain. For example, compounds that associate with EGFR may be compounds that act as competitive inhibitors, un-competitive inhibitors, and non-competitive inhibitors. Compounds that associate with EGFR also may be compounds that act as mediators or other regulatory compounds. In a preferred embodiment, compounds designed to associate with EGFR may be used therapeutically as inhibitors of EGFR activity.

The use of X-ray coordinates for structure determination, molecular design and selection and synthesis of compounds that associate with transmembrane proteins such as EGFR is known in the art. Published PCT application WO 95/35367 describes the use of X-ray structure coordinates to design, evaluate, synthesize and use compounds that associate with the active site of an enzyme. UK Patent Application 2306961A describes the use of X-ray coordinates in rational drug design. Published PCT application, WO 97/15588 describes the structural determination of a polypeptide using x-ray diffraction patterns as well as use of the coordinates and three-dimensional structure in finding compounds that associate with the polypeptide of interest.

In one aspect of the invention, the structural coordinates and structure may be compared to, or superimposed over, other similar molecules. Comparison of EGFR and other molecules for which a graphical structure or three-dimensional structural coordinates are available may be accomplished using available software applications, such as the Molecular Similarity application of QUANTA (Molecular Simulations, Inc., Waltham, Mass.).

Compounds that associate with EGFR also may be computationally evaluated and designed by screening and selecting chemical entities or fragments for their ability to associate with EGFR, and in a preferred embodiment, the EGFR kinase domain. Several methods may be used to accomplish this aspect of the invention. In one embodiment, one may visually inspect a computer-generated model of EGFR, and specifically the kinase domain, based on structural coordinates obtained as described herein. Computer generated models of chemical entities or specific chemical moieties can then be positioned in or around the catalytic domain and evaluated based on energy minimization and molecular dynamics, using, for example, available programs such as CHARMM or AMBER. Positioning of the chemical entity or fragment can be accomplished, for example with docking software such as Quanta and Sybyl. Additionally, known and commercially available computer programs may be used in selecting chemical entities or fragments. Once suitable chemical entities or fragments are selected, they may be assembled into a single compound, such as an inhibitor, mediator, or other regulatory compound. Known and commercially available model building software may assist in assembly.

In one aspect of the invention, compounds that associate with EGFR and specifically the EGFR kinase domain may be designed as a whole, rather than by assembly of specific chemical moieties or chemical entities. This embodiment may be carried out using computer programs such as LUDI (Biosym Technologies, San Diego, Calif.), LEGEND (Molecular Simulations, Burlington, Mass.), and Leap Frog (Tripos Associates, St. Louis, Mo.).

In an exemplary embodiment, a candidate compound is chosen based upon the desired sites of interaction with EGFR and the candidate compound in light of the sites of interaction identified previously from a study of EGFR kinase domain co-crystallized with a control compound. Once the specific interactions are determined, docking studies, using commercially available docking software, are performed to provide preliminary “modeled” complexes of selected candidate compound with EGFR.

Constrained conformational analysis can be performed using, for example, molecular dynamics (MD) to check the integrity of the modeled EGFR-inhibitor complex. Once the complex reaches its most favorable conformational state, the structure as proposed by the MD study is analyzed visually to ensure that the modeled complex complies with known experimental SAR/QSAR (structure-activity relationship/quantitative structure-activity relationship) based on measured binding affinities.

Other modeling techniques may also be used in accordance with the invention. Examples of these techniques are disclosed in Cohen et al., ((1990) Molecular Modeling Software and Methods of Medicinal Chemistry: Journal of Medical Chemistry, Vol. 33: 883-94) and Navia et al., ((1992) The Use of Structural Information in Drug Design: Current Opinions in Structural Biology, Vol. 2: 202-10), herein incorporated by reference in the entirety.

### Kits

This invention also contemplates use of EGFR proteins, fragments thereof, peptides, and their fusion products in a variety of diagnostic kits and methods for detecting the presence of EGFR. Typically the kit will have a compartment containing either a defined EGFR peptide or gene segment or a reagent which recognizes one or the other, e.g., inhibitor fragments or antibodies.

A kit for determining the binding affinity of a test compound to EGFR or a particular domain of EGFR (such as the kinase domain) will typically comprise a test compound, a labeled compound, e.g., a receptor or antibody having known binding affinity for EGFR, a source of EGFR (naturally occurring or recombinant), and a means for separating bound from free labeled compound, such as a solid phase for immobilizing EGFR. Once compounds are screened, those having suitable binding affinity to the EGFR can be evaluated using assays known in the art, to determine whether they act as agonists or antagonists to the receptor.

One embodiment of the invention provides a kit for determining the concentration of EGFR protein in a sample. Such a kit typically comprises a labeled compound, e.g., ligand, inhibitor or antibody, having known binding affinity for EGFR, a source of EGFR (naturally occurring or recombinant), and a means for separating the bound from free labeled compound, for example, a solid phase for immobilizing the EGFR. Reagents and instructions will also normally be provided.

Antibodies, including antigen binding fragments, specific for the EGFR or ligand fragments are useful in diagnostic applications to detect the presence of elevated levels of EGFR and/or its fragments. Such antibodies may allow diagnosis of the amounts of differently processed forms of the EGFR. Such diagnostic assays can employ lysates, live cells, fixed cells, immunofluorescence, cell cultures, body fluids, and further can involve the detection of antigens related to the ligand in serum, or the like. Various commercial assays exist, such as radioimmunoassay (RIA), enzyme-linked immunosorbentassay (ELISA), enzyme immunoassay (EIA), enzyme-multiplied immunoassay technique (EMIT), substrate-labeled fluorescent immunoassay (SLFIA), etc. For example, unlabeled antibodies can be employed by using a second antibody which is labeled and which recognizes the antibody to an EGFR protein or to a particular fragment thereof. Similar assays have also been extensively discussed in the literature. See, e.g., Harlow and Lane ((1988) Antibodies: A Laboratory Manual, CSH Press, NY; Chan (ed.)).

Anti-idiotypic antibodies may have a similar use in detecting the presence of antibodies against an EGFR, as such may be diagnostic of various abnormal states. For example, overproduction of EGFR may result in production of various immunological or other medical reactions which may be diagnostic of abnormal physiological states, e.g., in cell growth, activation, or differentiation. Anti-idiotypic antibodies can be used to detect such abnormal physiological states that are a downstream effect of EGFR overexpression.

Frequently, the reagents for diagnostic assays are supplied in kits, so as to optimize the sensitivity of the assay. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent. The reagents may be provided as a dry lyophilized powder; such reagents may be reconstituted in an aqueous medium, thus providing appropriate concentrations of reagents for performing the assay.

Many of the aforementioned constituents of the drug screening and the diagnostic assays may be used without modification, or may be modified in a variety of ways. For example, labeling may be achieved by covalently or non-covalently joining a moiety which directly or indirectly provides a detectable signal. In any of these assays, the protein, test compound, EGFR, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating the bound from the free ligand, or alternatively the bound from the free test compound. The EGFR can be immobilized on various matrices followed by washing. Suitable matrices include plastic such as an ELISA plate, filters, and beads. Methods of immobilizing the EGFR to a matrix include, without limitation, direct adhesion to plastic, use of a capture antibody, chemical coupling, and biotin-avidin. The last step in this approach involves the precipitation of ligand/receptor or ligand/antibody complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle, et al. ((1984) Clinical Chemistry, Vol. 30(9): 1457-61), and the double antibody magnetic particle separation as described in U.S. Pat. No. 4,659,678.

### EXAMPLES

### Example I

### Expression and Purification of the Kinase Domain

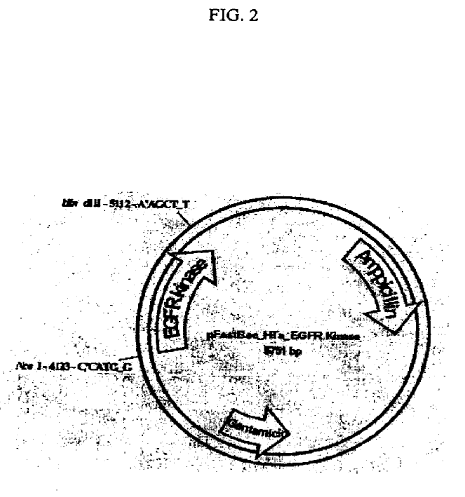

DNA encoding residues 672-998 of human EGFR was cloned into pFAST BAC HT (Invitrogen) using the NcoI and HindIII restriction sites (FIG. 2). The construct contains an N-terminal 6-His tag, a linker, and a cleavage site for the Tobacco Etch Virus protease (TEV). (MSYHHHHHHDYDIPTTENLYFQGAM). All mutations were introduced using the Quik-change site-directed mutagenesis kit (Stratagene). Sequences of all plasmids were confirmed by DNA sequencing.

Recombinant bacmid (Bac-to-Bac expression system, Gibco BRL) were transfected into Sf9 cells grown in suspension. Cells were harvested 2-3 days after infection by centrifugation at 4000×g and resuspended in a buffer containing 50 mM Tris, 5% glycerol, 1 mM DTT, and protease inhibitor cocktail (Roche), pH 8.0.

Cells were homogenized by French press in resuspension buffer and the lysate was centrifuged at 40000×g for 45 minutes. The supernatant was then loaded onto a 60 ml Q-Sepharose Fastflow column (Amersham) equilibrated in buffer A (50 mM Tris, 5% glycerol, and 15 mM β-mercaptolethanol, pH 8.0). Proteins were eluted using buffer A plus 1 M NaCl. The eluted protein was loaded onto a 1 ml Histrap column (Amersham) pre-equilibrated with buffer B (20 mM Tris, 500 mM NaCl, 5% glycerol, 20 mM imidazole, pH 8.0) and eluted using a gradient of imidazole (20-250 mM) after extensive wash with buffer B. The eluted proteins were either purified immediately using a 6 ml Uno-Q column (Bio-rad) to produce His-tagged kinase domains, or treated with the TEV protease overnight at 4° C. to remove the N-terminal His-tag before being subjected to Uno-Q purification for crystallization (see Example IV), analytical ultracentrifugation (see Example VI), and static light scattering (see Example VII).

Proteins were diluted 10-fold using buffer C (20 mM Tris, 20 mM NaCl, 5% Glycerol, and 2 mM DTT, pH 8.0) and loaded onto the Uno-Q column pre-equilibrated with buffer C. Proteins were eluted using a gradient of NaCl (20-500 mM). Fractions containing the EGFR protein were pooled, concentrated, and buffer exchanged into 20 mM Tris, 50 mM NaCl, 2 mM TCEP, pH 8.0. Proteins were concentrated to 10-30 mg/ml and flash-frozen in liquid nitrogen and stored at −80° C. Mass spectrometric analysis was used to confirm the identity of the proteins.

### Example II

### Preparation of Small Unilamellar Vesicles

DOPC and DOGS-NTA-Ni lipids in chloroform (Avanti Polar Lipids, Inc) were mixed in a glass tube. A lipid film was formed upon removing chloroform under a stream of argon gas, followed by putting the tube under vacuum for at least 3 hours.

Rehydration buffer (10 mM MgCl₂, 20 mM Tris, pH 7.5) was added to the lipid film and incubated for at least three hours. Intermittent vigorous vortexing during the incubation was applied to convert the lipid film into large, multilamellar vesicles.

The multilamellar vesicles were then forced through a polycarbonate filter (pore size: 100 nm) 21-41 times using a mini extruder (Avanti Polar Lipids, Inc) to yield homogenous small unilamellar vesicles.

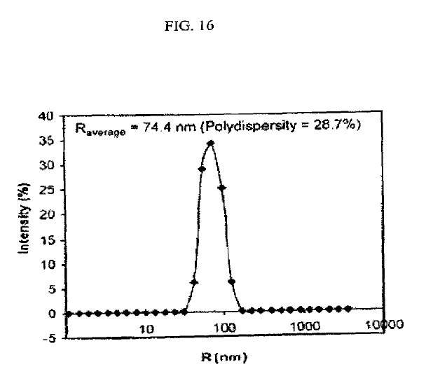

The diameter of the vesicles was measured by static light scatting to be in a range from 100-200 nm. (FIG. 16).

### Example III

### Kinase Assay in Solution and with Vesicles

A continuous enzyme-coupled kinase assay was performed to measure the kinase activity of the proteins as described in Barker et al., ((1995) Biochemistry, Vol. 34(54): 14843-51), with modifications, as described herein. The ATP concentration was kept to 0.5 mM.

The buffer used contained 10 mM MgCl₂, 20 mM Tris, and pH 7.5. Replacement of MgCl₂ by MnCl₂ in the assays resulted in a substantial increase of the catalytic activity of the kinase domain, as noted previously (Mohammadi et al., (1993) Biochemistry (34):8742-8; Wedergaertner and Gill, (1989) Journal of Biological Chemistry 264(19):11346-53). The substrate peptide was derived from the region spanning Y1173 in EGFR (TAENAEYLRVAPQ). All proteins used in this assay contained the N-terminal (His)₆ tag unless otherwise noted.

The protein concentrations of the EGFR kinase domain used in the assay ranged from 3.5 to 14 μM. The total concentration of the DOGS-NTA-Ni in the bulk solution was kept to 12.5 μM in all assays with DOG-NTA-Ni-containing vesicles. For assays of the kinase domain attached to vesicles, the protein and vesicles were preincubated at 4° C. for ˜5 min.

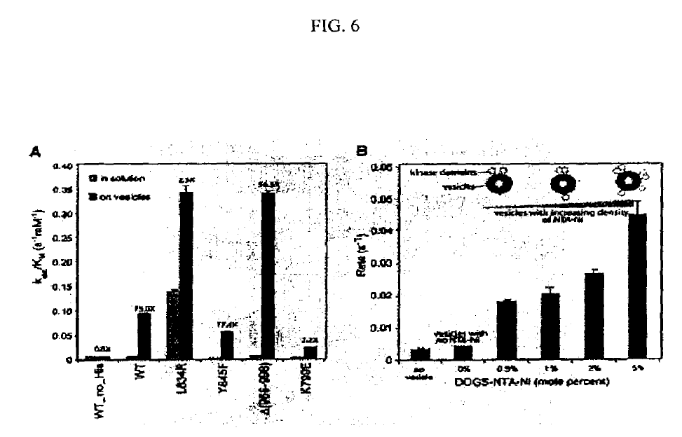

The wildtype EGFR kinase domain was mixed with vesicles containing 0, 0.5, 1, 2 and 5 mole percent of DOGS-NTA-Ni prior to the start of the assay. The final concentration of the protein in the assay was 3.5 μM. The substrate peptide concentration used in these assays was 1 mM. A sample of the kinase domain in the absence of lipid vesicles was also assayed using the same setup as a control. (FIG. 6B).

For comparing the specific activity of the wildtype and various mutant forms of the EGFR kinase domain in the presence and absence of lipid vesicles, the density of DOGS-NTA-Ni on lipid vesicles was kept at 5 mole percent. Preliminary experiments using the substrate peptide at various concentrations showed that the value of KM for the wildtype kinase domain and this substrate peptide was greater than 4 mM. Due to this high value of K_{M}, the values of K_{M} and k_{cat} were not measured directly. Instead, the value of k_{cat}/K_{M} was derived from a linear fit to the data obtained, using concentrations of the peptide that are much lower than the estimated value of K_{M} (V=[S]Vₘₐₓ/(K_{M}+[S]), V˜(Vₘₐₓ/K_{M})[S] when [S]<<K_{M}, k_{cat}=Vₘₐₓ/amount of the enzyme, where V and Vₘₐₓ are the initial velocity and maximum initial velocity, respectively. (FIG. 6A).

### Example IV

### Crystallization and Structure Determination

Two ATP analog conjugates were synthesized as described (Parang et al., 2001). The peptide sequences were AEEEIYGEFEAKK (the Src substrate peptide, Levinson et al., 2006) and ENAEYLRVAPQK (from a region that spans Tyr1173 in EGFR). The wildtype kinase domain with the His-tag removed (containing an N-terminal tri-peptide with sequence “GAM” from the vector and residues 682-998 from EGFR) at 6 mg/ml was co-crystallized with each of the synthesized peptides.

Diffraction data were collected at −170° C. at Beamlines 8.2.2, 8.3.1, and 12.3.1 at the ALS and processed using HKL2000 suite. The high R_{sym} values of the data for the active structures at the highest resolution shell are partially due to the high redundancy of the data. The data are included for refinement since they contain valid information as judged by the I/σ values and the quality of electron densities. The data for the inactive structure may be compromised by multiple lattices and high mosaicity in the diffraction pattern, which underlies the high free R value of the final model of the inactive structure.

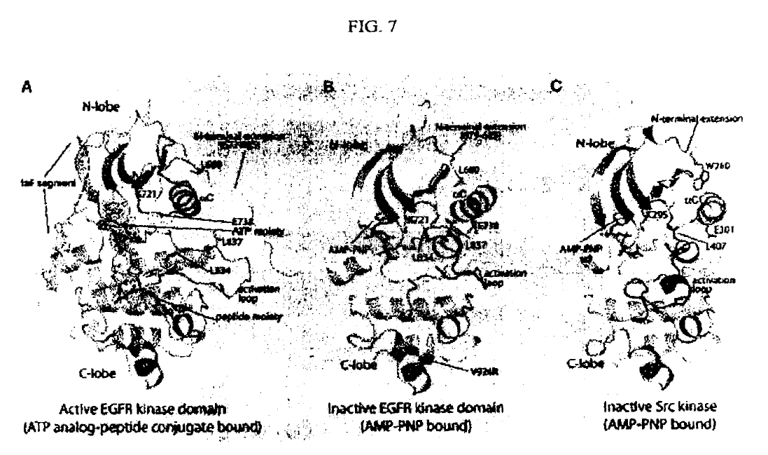

The original structures of active (PDB ID: 1M14) (Stamos et al., (2002) The Journal of Biological Chemistry, Vol. 277(48): 46265-72) and inactive (PDB ID: 1XKK) EGFR kinase domain was used as the starting model for solving the active and inactive structures. The structures were refined by iterative structural refinement using the program CNS and manual model building using the program O. (Brunger et al. (1998) Acta Crystallographica, Section D Biological Crystallography, Vol. 54(Pt. 5): page 905-21). The ATP analog-peptide conjugate and the AMP-PNP molecules were built after the free R-value dropped below 32%. (See FIG. 7).

### Example V

### Cell-Based Signaling Analysis

The EGFR full-length gene with a fragment encoding an N-terminal FLAG antibody recognition sequence (DYKDDDDK) inserted between the 24-residue signal peptide and the mature protein was amplified by PCR and cloned into the pcDNA3.1 vector (BD Biosciences) using XhoI and XbaI restriction enzymes.

Mutations were generated by using the Quickchange site-directed mutagenesis kit. All plasmids used for transfection were prepared using the HiSpeed Plasmid Midi kit (Qiagen) and the sequences were confirmed by DNA sequencing prior to use.

NIH3T3 cells (which express low levels of endogenous EGFR that are undetectable by Western blot; Bishayee et al., 1999) were cultured in Dulbecco's modified Eagle's medium supplemented with 10% fetal bovine serum, streptomycin/penicillin, sodium pyruvate, and nonessential amino acids (all from Gibco) at 37° C. with 5% CO₂.

Cells were plated and cultured overnight in 6-well plates in the same medium without antibiotics for transfection. Cells were transfected using Fugene 6 (Roche) according to the manufacturer's instructions with a DNA:Fugene 6 ratio of 1.5 μg:4.5 μl when cells reacted ˜50% confluency.

Cells were cultured for ˜36 hours after transfection and serum-starved for ˜12 hours before ligand stimulation and harvesting. Ligand stimulation of cells was performed using 50 ng/ml EGF (PeproTech, Inc.,) at 37° C. for 5 minutes. Cells were lysed in a buffer containing 50 mM Tris, 150 mM NaCl, 1 mM EDTA, 1 mM Na₃VO₄, 1 mM NaF, 1% Triton X-100, and a protease inhibitor cocktail (Roche), pH 7.5.

The lysates were centrifuged at 14,000×g for 10 minutes to remove insoluble material. The supernatants were collected and the protein concentrations were determined using the Bradford protein assay (Bio-Rad) for normalizing the total amount of proteins loaded onto the gels. Samples were run on SDS gels and subjected to Western blot analysis. The total amount of EGFR was monitored using an anti-FLAG antibody (Sigma). The levels of phosphorylation of EGFR at three sites were monitored using anti-EGFR antibodies specific for phosphorylation at Tyr1045 (Cell Signaling), Tyr1068 (Cell Signaling), and Tyr1173 (Santa Cruz). (FIG. 9B and FIG. 19).

### Example VI

### Analytical Ultracentrifugation

Sedimentation equilibrium experiments were performed using wildtype EGFR kinase domain protein (with the N-terminal His-tag removed) in 100 mM NaCl, 1 mM TCEP, 10 mM Tris, pH 8.0 at protein concentrations of 13.3 μM, 26.6 μM, and 53 μM in a Beckman XL-I ultracentrifuge using an AN-60 Ti rotor at 20° C., 20000 rpm.

Scans at 280 nm and 300 nm were taken every three hours and equilibrium was assumed to have been reached if two consecutive scans were identical. Data were collected at both wavelengths in a radial step mode with 0.001 cm step-size and 20-point averages. Data analysis and Monte Carlo analysis were performed using the software Ultrascan. The partial specific volume and buffer density of the protein were calculated to be 0.74 ml/g and 1.003 g/ml respectively using the same software.

Five of the six data sets taken at the three protein concentration and two wavelengths were fitted globally to multiple models. The data set taken at 300 nm for the sample at 13.3 μM was excluded from the fitting because the signals were too weak to be fit reliably. A one-species ideal model with a molecular weight of 37890 Da was found to be most appropriate, very close to the molecular weight calculated from the protein sequence (37516 Da). Consequent Monte Carlo analysis suggested that the molecular weight was within the range of 37476-38296 Da with 99% confidence. (FIG. 15A).

### Example VII

### Multi-Angle Static Light Scattering

The wildtype EGFR kinase domain with the N-terminal His-tag removed at 1-2 mg/ml (27-53 μM) concentration was loaded on to a KW-803 size exclusion column pre-equilibrated in 10 mM NaHPO₄—NaH₂PO₄, 100 mM NaCl, pH 7.5 at a flow rate of 0.4 ml/min. The protein eluted from the chromatography system was detected by a coupled 18-angle light scattering detector and refractive index detector with a data collection interval of 0.5 seconds. Data analysis was performed using the program ASTRA, which yielded a molecular weight for the EGFR kinase domain of 39500 Da. (FIG. 15B).

### Example VIII

### Western Blot

The levels of phosphorylation of EGFR at three sites were monitored using anti-EGFR antibodies specific for phosphorylation at Tyr1045 (Cell Signaling), Tyr1068 (Cell Signaling) and Tyr1173 (Santa Cruz). The total amount of EGFR in the samples was monitored using an anti-FLAG antibody (Sigma). All Western blots, except those from (FIG. 19), were performed as follows: Anti-EGFR (phospho-Tyr1068) and the FLAG epitope were analyzed separately by transferring protein bands from 8% SDS gels to PVDF membranes. Subsequently, the membranes were stripped in a buffer containing 2% SDS, 100 mM β-mercaptoethanol, 50 mM Tris, pH 6.8. (See FIG. 9, FIG. 10, and FIG. 12). The membranes used for the phospho-Tyr1068 Western blot was reblotted with anti-EGFR (phospho-Tyr1045), and that originally used for the anti-FLAG blot was reblotted with anti-EGFR (phospho-Tyr1173). Western blots shown in (FIG. 19) were done using four separate gels.

What is claimed is:

1. A method of targeted drug discovery, said method comprising:
a. contacting an isolated EGFR kinase domain with a test compound;
b. detecting an increase in EGFR kinase domain activity,
thereby identifying said test compound as an inhibitor of EGFR.

2. The method of claim 1, wherein said test compound binds in a hydrophobic pocket between helix C of said EGFR kinase domain and the main body of said EGFR kinase domain.

3. A pharmaceutical composition comprising said test compound of claim 1, wherein said test compound is combined with at least one pharmaceutically acceptable carrier.

4. A method for screening for potential inhibitors of EGFR activation comprising:
a) attaching an isolated polypeptide corresponding to an EGFR kinase domain to a lipid vesicle surface to form a conjugated polypeptide;
b) determining activity of said conjugated polypeptide;
c) contacting said conjugated polypeptide with a test compound
following c), determining activity of said conjugated polypeptide; and
d) comparing said activity of b) with said activity of c), wherein when said activity determined in c) is less than said activity determined in b) identifies said test compound as an inhibitor of EGFR activation.

5. The method of claim 4, wherein said test compound binds in a hydrophobic pocket between helix C of said EGFR kinase domain and the main body of said EGFR kinase domain.

6. A method for inhibiting EGFR activation, said method comprising contacting an EGFR kinase domain with a test molecule that interacts with said EGFR kinase domain, said contacting between said EGFR kinase domain and said test molecule serving to preventing interaction of N-lobe of said EGFR kinase domain with C-lobe of said EGFR kinase domain, thereby inhibiting EGFR activation.


SEQUENCE LISTING (11 sequences)

SEQ ID NO: 1 - protein, 55 aa, homo sapiens
EDRPPKVPPREPLSPSNSRTPSPKSLPSYLNGVMPPTQSFAPDPKYVSSKALQRQ

SEQ ID NO: 2 - protein, 26 aa, Homo sapiens
KSLPSYLNGVMPPTQSFAPDPKYVSS

SEQ ID NO: 3 - protein, 26 aa, Homo sapiens
KSLPSYLNGVMPPTQSFAPDPKYVSS

SEQ ID NO: 4 - protein, 25 aa, Homo sapiens
SYLNGVMPPTQSFAPDPKYVSSKAL

SEQ ID NO: 5 - protein, 16 aa, Homo sapiens
MPPTQSFAPDPKYVSS

SEQ ID NO: 6 - protein, 327 aa, Homo sapiens
GEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQG

SEQ ID NO: 7 - protein, 13 aa, Homo sapiens
TAENAEYLRVAPQ

SEQ ID NO: 8 - protein, 12 aa, Homo sapiens
ENAEYLRVAPQK

SEQ ID NO: 9 - protein, 13 aa, Homo sapiens
AEEEIYGEFEAKK

SEQ ID NO: 10 - DNA, 5751 bp, Homo sapiens
gacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcagaccagccgcgtaacctggcaaaatcggttacggttgagtaataaatggatgccctgcgtaagcgggtgtgggcggacaataaagtcttaaactgaacaaaatagatctaaactatgacaataaagtcttaaactagacagaatagttgtaaactgaaatcagtccagttatgctgtgaaaaagcatactggacttttgttatggctaaagcaaactcttcattttctgaagtgcaaattgcccgtcgtattaaagaggggcgtggccaagggcatggtaaagactatattcgcggcgttgtgacaatttaccgaacaactccgcggccgggaagccgatctcggcttgaacgaattgttaggtggcggtacttgggtcgatatcaaagtgcatcacttcttcccgtatgcccaactttgtatagagagccactgcgggatcgtcaccgtaatctgcttgcacgtagatcacataagcaccaagcgcgttggcctcatgcttgaggagattgatgagcgcggtggcaatgccctgcctccggtgctcgccggagactgcgagatcatagatatagatctcactacgcggctgctcaaacctgggcagaacgtaagccgcgagagcgccaacaaccgcttcttggtcgaaggcagcaagcgcgatgaatgtcttactacggagcaagttcccgaggtaatcggagtccggctgatgttgggagtaggtggctacgtctccgaactcacgaccgaaaagatcaagagcagcccgcatggatttgacttggtcagggccgagcctacatgtgcgaatgatgcccatacttgagccacctaactttgttttagggcgactgccctgctgcgtaacatcgttgctgctgcgtaacatcgttgctgctccataacatcaaacatcgacccacggcgtaacgcgcttgctgcttggatgcccgaggcatagactgtacaaaaaaacagtcataacaagccatgaaaaccgccactgcgccgttaccaccgctgcgttcggtcaaggttctggaccagttgcgtgagcgcatacgctacttgcattacagtttacgaaccgaacaggcttatgtcaactgggttcgtgccttcatccgtttccacggtgtgcgtcacccggcaaccttgggcagcagcgaagtcgaggcatttctgtcctggctggcgaacgagcgcaaggtttcggtctccacgcatcgtcaggcattggcggccttgctgttcttctacggcaaggtgctgtgcacggatctgccctggcttcaggagatcggaagacctcggccgtcgcggcgcttgccggtggtgctgaccccggatgaagtggttcgcatcctcggttttctggaaggcgagcatcgtttgttcgcccaggactctagctatagttctagtggttggctacgtatactccggaatattaatagatcatggagataattaaaatgataaccatctcgcaaataaataagtattttactgttttcgtaacagttttgtaataaaaaaacctataaatattccggattattcataccgtcccaccatcgggcgcggatctcggtccgaaaccatgtcgtactaccatcaccatcaccatcacgattacgatatcccaacgaccgaaaacctgtattttcagggcgccatgggagaagctcccaaccaagctctcttgaggatcttgaaggaaactgaattcaaaaagatcaaagtgctgggctccggtgcgttcggcacggtgtataagggactctggatcccagaaggtgagaaagttaaaattcccgtcgctatcaaggaattaagagaagcaacatctccgaaagccaacaaggaaatcctcgatgaagcctacgtgatggccagcgtggacaacccccacgtgtgccgcctgctgggcatctgcctcacctccaccgtgcaactcatcacgcagctcatgcccttcggctgcctcctggactatgtccgggaacacaaagacaatattggctcccagtacctgctcaactggtgtgtgcagatcgcaaagggcatgaactacttggaggaccgtcgcttggtgcaccgcgacctggcagccaggaacgtactggtgaaaacaccgcagcatgtcaagatcacagattttgggctggccaaactgctgggtgcggaagagaaagaataccatgcagaaggaggcaaagtgcctatcaagtggatggcattggaatcaattttacacagaatctatacccaccagagtgatgtctggagctacggggtgaccgtttgggagttgatgacctttggatccaagccatatgacggaatccctgccagcgagatctcctccatcctggagaaaggagaacgcctccctcagccacccatatgtaccatcgatgtctacatgatcatggtcaagtgctggatgatagacgcagatagtcgcccaaagttccgtgagttgatcatcgaattctccaaaatggcccgagacccccagcgctaccttgtcattcagggggatgaaagaatgcatttgccaagtcctacagactccaacttctaccgtgccctgatggatgaagaagacatggacgacgtggtggatgccgacgagtacctcatcccacagcagggttagaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctaggagatccgaaccagataagtgaaatctagttccaaactattttgtcatttttaattttcgtattagcttacgacgctacacccagttcccatctattttgtcactcttccctaaataatccttaaaaactccatttccacccctcccagttcccaactattttgtccgcccacagcggggcatttttcttcctgttatgtttttaatcaaacatcctgccaactccatgtgacaaaccgtcatcttcggctactttttctctgtcacagaatgaaaatttttctgtcatctcttcgttattaatgtttgtaattgactgaatatcaacgcttatttgcagcctgaatggcgaatgg

SEQ ID NO: 11 - DNA, 5148 bp, Homo sapiens
acgttatcgactgcacggtgcaccaatgcttctggcgtcaggcagccatcggaagctgtggtatggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacagtattcatgtcccctatactaggttattggaaaattaagggccttgtgcaacccactcgacttcttttggaatatcttgaagaaaaatatgaagagcatttgtatgagcgcgatgaaggtgataaatggcgaaacaaaaagtttgaattgggtttggagtttcccaatcttccttattatattgatggtgatgttaaattaacacagtctatggccatcatacgttatatagctgacaagcacaacatgttgggtggttgtccaaaagagcgtgcagagatttcaatgcttgaaggagcggttttggatattagatacggtgtttcgagaattgcatatagtaaagactttgaaactctcaaagttgattttcttagcaagctacctgaaatgctgaaaatgttcgaagatcgtttatgtcataaaacatatttaaatggtgatcatgtaacccatcctgacttcatgttgtatgacgctcttgatgttgttttatacatggacccaatgtgcctggatgcgttcccaaaattagtttgttttaaaaaacgtattgaagctatcccacaaattgataagtacttgaaatccagcaagtatatagcatggcctttgcagggctggcaagccacgtttggtggtggcgaccatcctccaaaatcggatctggaagttctgttccaggggcccctgggatccaggcctcccaaagtaccgccaagagaacctttgtcaccgagtaactcgcgcacaccgagtcccaaaagccttccgtcttacctcaatggggtcatgcccccgacacagagctttgcccctgatcccaagtatgtcagcagcaaagcactgcaaagacagaacagcgaaggatctgccagttagctcgagcggccgcatcgtgactgactgacgatctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtataattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataaattccgacaccatcgaatggtgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacggattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtaacctatcccattacggtcaatccgccgtttgttcccacggagaatccgacgggttgttactcgctcacatttaatgttgatgaaagctggctacaggaaggccagacgcgaattatttttgatggcgttggaatt